(19) United States
(12) Patent Application Publication
(10) Pub. No.: US 2007/0123555 A1
(43) Pub. Date: May 31, 2007

(54) PREVENTION AND TREATMENT OF HEARING DISORDERS

(75) Inventors: Srinivas RAO, Encinitas, CA (US); Jay Kranzler, La Jolla, CA (US); Jeffery J. Anderson, San Diego, CA (US)

Correspondence Address: WILSON SONSINI GOODRICH & ROSATI, 650 PAGE MILL ROAD, PALO ALTO, CA, 94304-1050, US

(73) Assignee: Cypress Bioscience, Inc., San Diego, CA (US)

(21) Appl. No.: 11/536,538

(22) Filed: Sep. 28, 2006

Related U.S. Application Data
(60) Provisional application No. 60/722,784, filed on Sep. 30, 2005.

(51) Int. Cl. A61K 31/44; A01N 43/42

(52) U.S. Cl. 514/288

(57) ABSTRACT

Compositions, and methods of use thereof, are provided for the prevention, treatment or alleviation of symptoms of hearing are provided. The methods employ nicergoline, MMDL or MDL as the sole active pharmaceutical agent or a combination of nicergoline, MMDL or MDL and another pharmaceutical agent, such as an antioxidant, a NMDA antagonist, an SSRI or a combined SSRI/NMDA antagonist agent. The method involves the use of nicergoline, MMDL or MDL alone or in combination with another API to prevent, treat or ameliorate one or more symptoms of hearing loss.

### CROSS REFERENCE TO RELATED APPLICATIONS

This application claims benefit of priority under 35 U.S.C. §119(e) from U.S. provisional patent application 60/722,784, filed on Sep. 30, 2005, which is incorporated herein by reference in its entirety.

### FIELD OF THE INVENTION

This invention generally relates to methods and compositions for the pharmacological prevention and treatment of hearing disorders, such as hearing loss and tinnitus.

### BACKGROUND OF THE INVENTION

Hearing loss is a growing problem in the industrialized word, with varied and complex etiology. While some forms of hearing loss are clearly genetic in origin, others are either wholly or at least partially environmental in nature. Central, peripheral, or both types of, mechanisms can be involved in hearing loss. Among the forms of hearing loss are presbycusis, noise-induced hearing loss and drug-induced hearing loss.

Centrally, the decrease in neurotransmitters, such as serotonin (5-HT) can lead to perceived hearing loss. Cruz et al. have presented evidence that suggests that the use of citalopram can have a positive impact on auditory processes in the elderly. (Oswaldo Laercio M. Cruz et al, Serotonin Reuptake Inhibitors in Auditory Processing Disorders in Elderly Patients: Preliminary Results, Laryngoscope, 114, September 2004, 1656-1659.) However, Cruz et al. does not suggest combining S-citalopram with nicergoline or its metabolites.

Presbycusis (also presbyacusis) begins after age 20 but is usually significant only in persons over 65. Men are affected more often and more severely than women. Stiffening of the basilar membrane and deterioration of the hair cells, stria vascularis, ganglion cells, and cochlear nuclei may play a role in pathogenesis, and presbycusis appears to be related in part to noise exposure. It first affects the highest frequencies (18-20 kHz) and gradually begins to affect the 4- to 8-kHz range by age 55 to 65, although variation is considerable. Some persons are severely handicapped by age 60, and some are essentially untouched by age 90. The loss of high-frequency hearing makes discrimination of speech particularly difficult. Thus, many persons who have this type of hearing loss have difficulty understanding conversation, particularly when background noise is present, and complain that others mumble. Although speech reading (lip reading), auditory training for making maximum use of non-auditory clues, and amplification with a hearing aid are helpful, better therapeutic alternatives would be welcome. L-carnitine has been administered to rats in a model of presbycusis. A. Derin et al., The Effects of L-carnitine on Presbyacusis in the Rat Model, Clin. Otolaryngol., 2004, 29, 238-241. However, Derin et al. do not teach or suggest the combination of nicergoline or its metabolites with L-carnitine, either in compositions or in methods relating to hearing loss.

Noise-induced hearing loss can arise under either acute or chronic circumstances. Extremely loud sounds can give rise to sudden hearing loss. While such hearing loss not infrequent, hearing loss due to long-term exposure to excessive noise is more common.

Noise-induced hearing loss can give rise to multifarious problems. In addition to the inability to hear certain sounds, especially in the upper registers, one experiencing such hearing loss may also experience tinnitus, or ringing in the ears, which is characterized by abnormal sounds and auditory sensations that may persist for various lengths of time after cessation of auditory stimulation or in the absence of sound. Additionally, noise can mechanically irritate the inner ear, giving rise to an inflammatory response characterized by fluid buildup and concomitant dampening of sound transmission within the ear. Moreover, excessive noise can, directly or indirectly, cause damage to the 8^{th} nerve, giving rise to a neuronal type of hearing loss. In the earlier stages of neuronal hearing loss, the patient experiences a degradation of his ability to process sounds and speech. Thus, for instance, the patient may lose the ability to discriminate between certain words or to understand certain persons, especially those whose voices are in the upper or lower registers.

Another important type of hearing loss is drug-induced hearing loss. Ototoxic drugs include chemotherapeutic agents, such as antineoplastic agents and antibiotics. Other ototoxic drugs include loop-diuretics, quinines or a quinine-like compound, and salicylate or salicylate-like compounds.

Aminoglycosides are antibiotics that have been used for the treatment of Gram-negative bacterial infections and some aerobic Gram-positive bacterial infections. Despite their utility, however, they have serious side effects, including ototoxicity. Aminoglycoside ototoxicity is associated with the destruction of the sensory hair cells in organ of Corti of the cochlea of the inner ear. See Bates et al., “Aminoglycoside Ototoxicity,” Drugs of Today 39(4), 277-285 (2003)(incorporated herein by reference in its entirety).

Cisplatin is an antineoplastic agent that is commonly used in the treatment of cancer. Like many antineoplastic agents, however, cisplatin has several known and widely documented toxicities, including cytotoxicity. In particular, cisplatin treatment gives rise to hair cell degeneration in the organ of Corti. Although D-methionine has been suggested as a protectant for the cochlea during administration of cisplatin, effective methods of protecting against cisplatin-induced hearing loss have yet to be fully realized. See Campbell et al., “D-Methionine provides excellent protection from cisplatin ototoxicity in the rat,” Hearing Research 102, 90-98 (1996)(incorporated herein by reference in its entirety). Moreover, D-methionine is of extremely low potency.

Central auditory processing disorder (CAPD) is a deficit in the neural processing of auditory stimuli that is not due to higher order language, cognitive or related factors. While higher order cognitive-communication or language-related functions may be associated with CAPD, they are not included within the definition of CAPD. Nevertheless, CAPD may exacerbate or even give rise to one or more higher order cognitive-communication or language-related function disorders. Indeed, CAPD can lead to, or be associated with, one or more difficulties in learning, speech, language, social functioning. At present, there are no accepted pharmaceutical therapeutic approaches to treatment of CAPD.

There is a need for pharmaceuticals and methods for protecting the ear from damage by excessive noise. Additionally, there is a need for compositions and methods of treating a mammal to reduce, ameliorate or counteract one or more symptoms of noise-induced hearing loss, such as the abnormal sounds and auditory sensations associated with tinnitus. Additionally, there is a need for pharmaceuticals and methods of treating a mammal, such as a human, to restore hearing to the mammal by treating the effects of noise-induced hearing loss.

There is likewise a need for pharmaceuticals and methods for protecting the ear from damage by an ototoxic drug. Additionally, there is a need for compositions and methods of treating a mammal to reduce, ameliorate or counteract one or more symptoms of drug-induced hearing loss. Additionally, there is a need for pharmaceuticals and methods of treating a mammal, such as a human, to restore hearing to the mammal by treating the effects of drug-induced hearing loss.

### SUMMARY OF THE INVENTION

The foregoing and other needs are met by embodiments of the invention, which provide a method of preventing or treating a hearing disorder in a mammal, such as a human. The method includes administering to the mammal an amount of nicergoline, MMDL or MDL, either alone or in combination with one or more active pharmaceutical ingredients, sufficient to prevent or treat one or more hearing disorders. The additional pharmaceutical ingredients useful in combination with nicergoline, MMDL or MDL include: antioxidants (or spin trapping agents); norepinephrine and/or serotonin reuptake inhibitors; NMDA antagonists; SSRI/NMDA antagonists; dopamine releaser/NMDA antagonists; acetylcholine release inducer/antioxidant/NMDA antagonist/NERI; MAO-A inhibitor/SRI/antioxidant; calcium channel antagonists and SSRI or NSRI; or 5-HT SRI/NRI/ACE releaser/NMDA antagonists.

The foregoing and other needs are further met by embodiments of the invention, which provide compositions for preventing one or more hearing disorders in a mammal, such as a human. The compositions of the invention comprise nicergoline, MMDL or MDL alone in combination with one or more of the aforementioned second active pharmaceutical ingredients in an amount sufficient to provide hearing protective or hearing loss treating benefit to a mammal, such as a human.

The foregoing and other needs are further met by kits containing two or more active pharmaceutical ingredients for the treatment or prevention of hearing disorders in separate dosage forms. The first ingredient is selected from the group consisting of nicergoline, MMDL, MDL and a combination of two or more thereof. The second ingredient is selected from the group consisting of: antioxidants (or spin trapping agents); norepinephrine-serotonin reuptake inhibitors; NMDA antagonists; SSRFNMDA antagonists; dopamine releaser/NMDA antagonists; acetylcholine release inducer/antioxidant/NMDA antagonist/NERI; MAO-A inhibitor/SRI/antioxidant; calcium channel antagonists and SSRI or NSRI; or 5-HT SRI/NRI/ACE releaser/NMDA antagonists.

The present invention further meets the foregoing needs by providing a method of decreasing the variability associated with CYP2D6 polymorphism and increasing the efficacy of nicergoline therapy in humans. The method comprises administering to a human patient a therapeutically effective amount of a therapeutic composition comprising the nicergoline metabolite, MDL. The method is thus effective to treat or prevent at least one hearing disorder as described in more detail herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which

FIG. 1 shows that nicergoline protects cochlear ear explants from chemically-induced damage by cisplatin.

### DETAILED DESCRIPTION OF THE INVENTION

The invention provides compositions, methods and kits for prevention or treatment of hearing disorders. In the context of this invention, the conjunction “or,” unless otherwise qualified, is used in the inclusive sense. Thus, for example, “prevention or treatment” means “prevention, treatment or both.”

Therapeutic compositions according to the present invention comprise nicergoline, MMDL, MDL, or a combination of two or more thereof. In some embodiments, the therapeutic compositions according to the invention further comprise one or more active pharmaceutical ingredients (APIs) as described in more detail below. Thus, unless otherwise modified herein, the phrase “therapeutic composition according to the invention” refers to a composition comprising one or more members of the group consisting of nicergoline, MMDL and MDL.

A “hearing disorder” is a central or peripheral hearing disorder, such as hearing loss, tinnitus or hyperacusis. Hearing loss includes conductive hearing loss and sensorineural hearing loss. Types of sensorineural hearing loss include: presbycusis, noise-induced hearing loss (NIHL), drug-induced hearing loss (DIHL) and central auditory processing disorder (CAPD).

Prevention of a hearing disorder means providing partial or complete protection against a hearing disorder. Thus, prevention of a hearing disorder includes provision of partial or complete protection against hearing loss, tinnitus or hyperacusis.

Treatment of a hearing disorder means providing palliation, amelioration or reversal of a hearing disorder. Thus, treatment of a hearing disorder includes provision of partial or complete palliation, amelioration or reversal of hearing loss, tinnitus or hyperacusis.

Within the context of the present invention, prevention of hearing loss means protection against hearing loss, such as noise-induced hearing loss. In particular, prevention of hearing loss includes protection against damage to hair cells within the cochlea of the inner ear. By protecting the hair cells from noise-induced damage, the invention prevents loss of hearing by the patient.

Within the context of the invention, treatment of hearing loss means reducing hearing loss or ameliorating one or more symptoms of hearing loss, especially noise-induced hearing loss. Thus, treatment of hearing loss includes: improving hearing; reducing transmission of abnormal sounds and auditory sensations associated with tinnitus; reducing fluid accumulation associated with disorders of the inner ear; facilitating central auditory processing of sounds in the inner ear; improving voice recognition or processing; and/or ameliorating one or more additional symptoms of hearing loss.

When used herein, the term patient means a mammal to which a therapeutic composition according to the invention is administered in order to elicit a therapeutic effect. The term mammal includes humans, as well as apes, monkeys, dogs, cats and rodents. A therapeutic effect in this sense includes prophylaxis, treatment and/or both. The person skilled in the art will recognize that the therapeutic effect will vary depending upon the desired outcome of administering the therapeutic compositions according to the invention.

The invention provides compositions for protection against hearing loss, treatment of hearing loss, or both. In some embodiments, the therapeutic compositions according to the invention comprise nicergoline, MMDL or MDL as the sole active pharmaceutical ingredient. In such embodiments, nicergoline, MMDL or MDL may be admixed with one or more inactive ingredients. Nicergoline, MMDL or MDL may also be coated with one or more inactive ingredients. In some embodiments, nicergoline, MMDL or MDL may be mixed with one or more other inactive ingredients and coated with one or more inactive ingredients. In some embodiments, nicergoline, MMDL or MDL and at least one inactive ingredient may be separated (segregated) from one another to prevent their admixture. Such segregation may be effected in order to ensure release of one ingredient before the other, to prevent reaction between the ingredients or both. Inactive ingredients useful in various embodiments of the invention are discussed in more detail below.

In other embodiments, a therapeutic composition according to the invention comprises nicergoline, MMDL or MDL in combination with another active pharmaceutical ingredient in the same dosage form. In this context “in combination” means that nicergoline, MMDL or MDL and the other active pharmaceutical ingredient are together in the same dosage form, such that they may be transported, dispensed and administered to the patient in a single convenient dosage form. Exemplary dosage forms are discussed in more detail below.

In such embodiments, the therapeutic composition according to the invention comprises nicergoline, MMDL or MDL admixed with one or more active pharmaceutical ingredients. Nicergoline, MMDL or MDL may also be coated with one or more active pharmaceutical ingredients. In some embodiments, nicergoline, MMDL or MDL may be mixed with one or more other active pharmaceutical ingredients and coated with one or more active or inactive pharmaceutical ingredients. In some embodiments, nicergoline, MMDL or MDL and at least one active pharmaceutical ingredient may be separated (segregated) from one another to prevent their admixture. Such segregation may be effected in order to ensure release of one ingredient before the other, to prevent reaction between the ingredients or both. Active pharmaceutical ingredients useful in various embodiments of the invention are discussed in more detail below.

I. Compositions

The present invention provides a therapeutic composition comprising one or more members of the group consisting of nicergoline, MMDL and MDL, or pharmaceutically acceptable salts, polymorphs, or stereoisomers thereof. It is to be understood that reference to nicergoline, MMDL and MDL herein, unless otherwise modified, also embraces pharmaceutically acceptable salts and/or polymorphs thereof, and that such is intended even without specifically reciting salts or polymorphs unless otherwise stated.

In addition, the invention provides therapeutic compositions comprising a combination of one or more of nicergoline, MMDL or MDL with an additional active pharmaceutical agent. Exemplary additional active pharmaceutical agents to be used in combination with nicergoline, MMDL and/or MDL include NMDA antagonists; SSI/NMDA antagonists; dopamine releaser/NMDA antagonists; acetylcholine release inducer/antioxidant/NMDA antagonist/NERI; MAO-A inhibitor/SRI/antioxidant; calcium channel antagonists and SSRI or NSRI; or 5-HT SRI/NRI/ACE releaser/NMDA antagonists.

A. APIs Useful for Treatment of Hearing Disorders

1. Nicergoline, MMDL and MDL

Nicergoline, MMDL and MDL are described in more detail below. Nicergoline is an ergot derivative that has been used for the treatment of senile dementia and as a vasodilator. MMDL and MDL are metabolites of nicergoline and are believed to be responsible for part or all of the biological activity of nicergoline. Thus, it is considered potentially advantageous to use nicergoline for the treatment or prevention of a hearing disorder. In addition it is believed that one or both of MMDL or MDL may be used in place of, or in admixture with, nicergoline for the treatment or prevention of a hearing disorder.

a. Nicergoline

Nicergoline ((8β-10-methoxy-1,6-dimethylergoline-8-methanol 5-bromo-3-pyridinecarboxylate (ester)) is an ergot derivative that has antioxidant activity as well as glutamate (Glu) reuptake stimulator activity. Without limiting the invention by theory, it is contemplated that antioxidants provide protection to the peripheral auditory system by sequestering or neutralizing the effects of toxic oxidants and free radical species in the peripheral auditory system. Furthermore, and without limiting the invention by theory, it is contemplated that glutamate is a mediator of noise-induced damage to the hair cells of the inner ear; blocking N-methyl-D-aspartate (NMDA) receptors provides protection against the toxic effects of glutamate. Nicergoline has been used as a vasodilator, as well as in the treatment of senile dementia.

Nicergoline was initially developed as a vasodilator and it lowers vascular resistance, thereby increasing arterial blood flow, in the brain. It is commercially available from Pfizer Inc. (Successor to Upjohn Corporation). The chemical structure of nicergoline is:

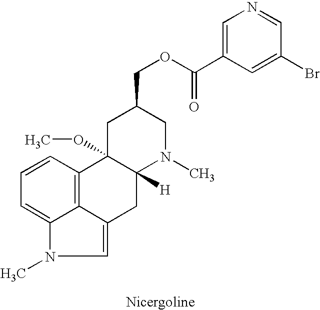

b. MMDL

MMDL (1-methyl-10-α-methoxy-9,10-dihydrolysergol (MMDL) is a metabolite of nicergoline formed by the hydrolytic cleavage of 5-bromobenzoate moiety from the 8-methoxy moiety of nicergoline. Its chemical structure is:

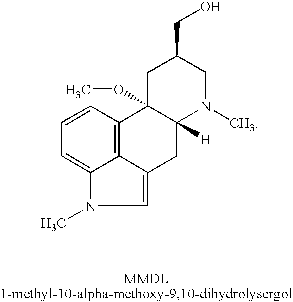

c. MDL

MDL (10-α-methoxy-9,9-dihydrolysergol) is a metabolite of nicergoline formed by the enzymatic demethylation of the 1-nitrogen of MMDL. Bottiger et al., “Involvement of CYP2D6 but not CYP2C19 in nicergoline metabolism in humans,” Br. J. Clin. Pharmacol., 42, 707-711 (1996). Several mutant alleles of CYP2D6, also known as dextromethorphan O-demethylase, have been identified. In particular, individuals having normal CYP2D6 function who have been dosed with nicergoline tend to have almost no detectable nicergoline in blood plasma, and very little MMDL as well, the vast majority of nicergoline being converted to MMDL, and the vast majority of MMDL being converted to MDL by CYP2D6. In contrast, poor metabolizers, i.e. those who express mutant alleles of CYP2D6 that have low demethylation activity, tend to have relatively high concentrations of MMDL and concomitantly low concentration of MDL in plasma. It is considered that poor metabolizers also tend to respond poorly to nicergoline therapy. Thus, it is considered that direct administration of MDL, circumventing the CYP2D6-mediated mechanism for N1-demethylation of nicergoline, will improve patient response to therapy directed at treating or preventing hearing disorders. In particular cases, the practitioner may first determine whether the patient expresses a mutant allele of the CYP2D6 gene, and if so, administer to the patient MDL instead of, or in addition to, nicergoline. In other embodiments, the practitioner may administer MDL to the patient, thereby avoiding the CYP2D6 mechanism altogether, and thereby reducing the clinical variability in response to therapy associated with polymorphism in the CYP2D6 gene.

The chemical structure of MDL is:

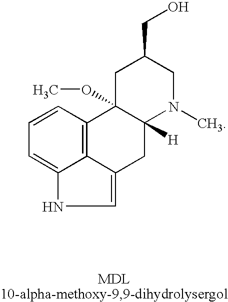

A therapeutic composition comprising nicergoline, MMDL and/or MDL, or pharmaceutically acceptable salts, polymorphs, or stereoisomers thereof, may further comprise one or more of the following active pharmaceutical ingredients: antioxidants/spin-trapping agents; NMDA antagonists; SSRI/NMDA antagonists; dopamine releaser/NMDA antagonists; compounds having combined acetylcholinesterase inducing, antioxidant, N-methyl-D-aspartate antagonist and norepinephrine reuptake inhibiting activity; compounds having combined monamineoxidase-A inhibiting, serotonin reuptake e inhibiting and antioxidant activity; norepineplrine and serotonin reuptake inhibitor/low affinity NMDA antagonists; combinations of calcium channel antagonists and SSRI or NSRI agents; compounds having 5-HT serotonin reuptake inhibiting, norepinephrine reuptake inhibiting, acetylcholine releasing and N-methyl-D-aspartate antagonist activity; or pharmaceutically acceptable salts, polymorphs, or stereoisomers thereof. It is to be understood that, unless otherwise modified, reference to an active pharmaceutical ingredient embraces, where applicable, related pharmaceutically acceptable salts, polymorphs and stereoisomers, and that reference to the API is intended, without further specification, to include the pharmaceutically acceptable salts, polymorphs and stereoisomers, without the need to specifically state as much with respect to each API. The additional active pharmaceutical ingredients are discussed in more detail below under subheadings 2-10.

2. Antioxidants and/or Spin-Trapping Agents

Antioxidants, such as allopurinol, glutathione, methionine and L-camitine reduce noise-induced damage to hair cells of the inner ear. These agents bind to or metabolize reactive oxygen species and provide protection against the damage induced by these toxic mediators. Nicergoline, MMDL, MDL, or combinations thereof, may be combined with one or more antioxidants to produce additive or synergistic effects. In particular nicergoline, MMDL or MDL may be combined with one or more antioxidants selected from the group consisting of: allopurinol, glutathione, methionine, L-camitine, ebselen, AEOL-10150 and mixtures of two or more thereof, or pharmaceutically acceptable salts, polymorphs or stereoisomers thereof.

a. Allopurinol

Allopurinol or 1,5-dihydro-4H-pyrazolo[3,4-d]pyrimidin-4-one is an antioxidant in the class of compounds known as xanthine oxidase inhibitors and is known as a pharmaceutical for the treatment of hyperuricemia and chronic gout.

b. Glutathione

Glutathione or N-(N-L-γ-glutamyl-L-cysteinyl)glycine is an antioxidant tripeptide. Glutathione is a very efficient antioxidant, as it is present in vivo almost exclusively in its reduced state due to the almost ubiquitous presence of the constitutively expressed enzyme glutathione reductase. Oxidative stress, which tends to oxidize glutathione to its dimeric form (cystinyl), also induces expression of glutathione reductase, which catalyzes the reduction of glutathione to its monomeric form (cysteinyl). Thus, glutathione acts as an efficient antioxidant in living tissue.

c. Methionine

Methionine or (S)-2-amino-4-(methylthio)butanoic acid is known as a hepatoprotectant, an antidote for acetaminophen poisoning and as a urinary acidifier.

d. L-Carnitine

L-carnitine, or 3-hydroxy-4N-trimethylaminobutryic acid, is a biological isomer of carnitine, which is a non-toxic compound that has an equal LD₅₀ value with other amino acids. It is known to stimulate fatty acid oxidation in the liver, heart and skeletal muscles. Carnitine is also a scavenger of superoxide ion and decreases free radical synthesis by inhibiting xanthine oxidase activity. Carnitine is known to have other effects in biological systems. For example, camitine plays a role in the active transportation of long-chain fatty acids into the mitochondrial matrix and intramitochondrial acyl-CoA/CoA regulation; hence carnitine is essential for mitochondrial oxidation of long-chain fatty acids. In addition, carnitine increases the proliferative response of human lymphocytes to mitogenic stimulation and polymorphonuclear chemotaxis, stabilizes the cell membrane and increases Ca²⁺ transport.

e. Ebselen

Ebselen (2-phenyl-1,2-benzisoselenazol-3(2H)-one) is a selenium-based inhibitor of protein kinase C, NADPH, 5-lipoxygenase, cyclooxygenase (COX) and NADPH oxidase, which has the following structure:

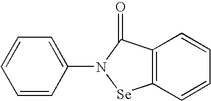

Ebselen has been shown to have anti-inflammatory and neuroprotective activity. It has also been shown to be an inhibitor of free radical-induced apoptosis, as well as an inhibitor of oxidative modifications of low density lipoproteins (LDL). It is commercially available in 1, 5 and 10 mg tablets.

f. Antioxidant Porphyrins (AEOL-10150)

Antioxidant porphyrins like AEOL-10150 [manganese (III) meso-tetrakis (di-N-ethylimidazole)porphyrin] are metalloporphyrins having potent in vitro superoxide dismutase and catalase activity. AEOL-10150 is a small molecule catalytic antioxidant that has shown the ability to scavenge a broad range of reactive oxygen species. As a catalytic antioxidant, AEOL-10150 mimics and thereby amplifies the body's natural enzymatic systems for eliminating these toxic compounds. Oxygen-derived free radicals are implicated in the pathogenesis of many diseases. In particular, antioxidant porphyrins have been shown to significantly reduce tissue damage in animal models of ALS, stroke, chronic obstructive pulmonary disease and mucositis caused by radiation therapy. The structure of AEOL-10150 is:

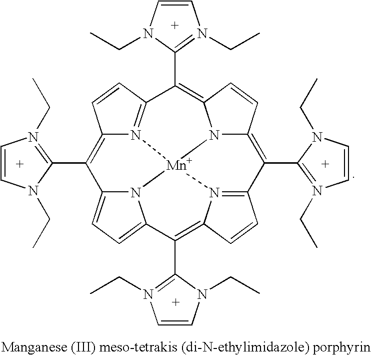

g. Combinations of Antioxidants

Specific combinations of antioxidants that may be combined with nicergoline, MMDL, MDL or a combination of two or more thereof, include: allopurinol and glutathione; glutathione and methionine; allopurinol and methionine; and allopurinol, glutathione and methionine. Another antioxidant combination that may be mentioned is ebselen with AEOL-10150. Other antioxidant combinations include one or more of allopurinol, glutathione or methionine with one or more of AEOL-10150, ebselen or both. In some embodiments, the combination of antioxidants are combined with nicergoline, MMDL or MDL in a single dosage form. In other embodiments, the antioxidants are administered in a dosage form separate from the dosage form containing nicergoline, MMDL or MDL.

3. NMDA Antagonists

In some embodiments, the therapeutic composition according to the invention comprise one or more of nicergoline, MMDL or MDL in combination with one or more N-methyl-D-aspartate (NMDA) antagonists, such as riluzole, caroverine, memantine, magnesium or mixtures thereof, or pharmaceutically acceptable salts, polymorphs or stereoisomers thereof.

NMDA antagonists block excitotoxic actions of glutamate within the inner ear. Glutamate is a mediator of noise-induced damage to the hair cells of the inner ear. Thus, blocking NMDA receptors provides protection against the toxic effects of glutamate. The combination of one or more of nicergoline, MMDL or MDL with an NMDA antagonist provides additive or synergistic effects in the prevention or treatment of a hearing disorder. Specific NMDA antagonists that may be combined with one or more of nicergoline, MMDL or MDL are described in more detail below.

a. Riluzole

Riluzole [2-amino-6-trofluoromethoxybenzothiazole] is a NMDA antagonist having wide-spectrum neuroprotective activity. It is also an anti-convulsant, which also has anti-ischemic and sedative properties. J. Wang et al., Riluzole Rescues Cochlear Sensory Cells From Acoustic Trauma in the Guinea Pig, Neuroscience, 111 (3), 2002, 635-648. Wang et al. have shown in a Guinea pig model that perfusion of riluzole into the cochlea via an osmotic minipump prevents mitochondrial damage and subsequent translocation of cytochrome c, DNA fragmentation and hair cell degeneration. Wang et al. failed to demonstrate oral dosing with riluzole, let alone combination therapy with riluzole and nicergoline, MMDL or MDL. The present invention contemplates a therapeutic composition, especially an oral therapeutic composition, comprising riluzole and one or more members of the group consisting of nicergoline, MMDL and MDL. As Wang et al. demonstrated that perfusion with riluzole can rescue the cochlea of Guinea pigs from noise-induced trauma, the invention contemplates that co-administration of riluzole and nicergoline and/or one of its metabolites offers additive or synergistic effects in the treatment or prevention of a hearing disorder.

b. Caroverine

Caroverine {1-[2-(diethylamino)ethyl]-3-[(4-methoxyphenyl)methyl]-2-(1H)-quinoxalinone}, is an NMDA antagonist and is known to be useful as an antispasmotic. Caroverine is an antagonist of two glutamate receptors. Zhiquiang Chen et al., Protection of Auditory Function Against Noise Trauma with Local Caroverine Administration in Guinea Pigs, Hearing Research, 197 (2004), 131-136. Chen et al. have presented data that demonstrate protection of cochlea from noise trauma in a guinea pig model. Thus, it is contemplated that a therapeutic composition according to the present invention comprising caroverine will protect the cochlea from noise trauma. As caroverine is an NMDA antagonist, it is expected that other NMDA antagonists will work together with nicergoline, MMDL and/or MDL to protect provide a therapeutic effect to the mammal. In particular embodiments, it is contemplated that such compositions will protect a mammal, such as a human, from noise-induced hearing loss.

c. Memantine

Memantine or 3,5-dimethyltricyclo[3.3.1.1]decan-1-amine is an NMDA antagonist and is know to be useful as a muscle relaxant. It is contemplated that a therapeutic composition according to the invention comprising memantine will demonstrate additive or synergist effects in the treatment or prevention of a hearing disorder.

d. Magnesium

Magnesium (Mg²⁺) is an essential factor in regulating cellular membrane permeability, neuromuscular excitability and energy production. Magnesium has been shown to antagonize the N-methyl-D-aspartate receptor. Daily ingestion of 122 mg of magnesium for 10 days was shown to protect against noise-induced temporary threshold shift. Attias et al., Reduction in noise-induced temporary threshold shift in humans following oral magnesium intake, Clin. Otolaryngol., 2004, 29, 635-641, which is incorporated herein by reference. Daily ingestion of 6.7 mmol of magnesium aspartate showed a significant effect on permanent threshold shift as compared to placebo (sodium aspartate) in normal, healthy volunteers exposed to rifle noise six days per week for eight weeks. Attias et al., Oral Magnesium Intake Reduces Permanent Hearing Loss Induced by Noise Exposure, Am. J. Otolaryngology, Vo. 15, no 1, (January-February), 1994, 26-32, which is incorporated herein by reference. Magnesium has also been used to treat sudden sensorineural hearing loss (SSNHL). Nageris et al., Magnesium Treatment for Sudden Hearing Loss, Ann. Otol. Rhinol. Laryngol. 113, 2004, 672-675, which is incorporated by reference.

e. Combinations of NMDA Antagonists

Combinations of NMDA antagonists include: riluzole and caroverine; caroverine and memantine; riluzole and memantine; riluzole, caroverine and memantine; riluzole and magnesium; caroverine and magnesium, memantine and magnesium; riluzole, caroverine and magnesium; caroverine, memantine and magnesium; riluzole, memantine and magnesium; and riluzole, caroverine, memantine and magnesium. The present invention contemplates therapeutic compositions, especially oral compositions, comprising nicergoline, MMDL and/or MDL in combination with one or more of these NMDA antagonists.

4. SSRI/NMDA Antagonists

Selective serotonin reuptake inhibitors (SSRIs) enhance synaptic levels of serotonin in the brain and enhance hearing by improving auditory processing, increasing the signal: noise ratio of environmental sounds, and by heightening attention. Examples of SSRIs include fluoxetine, sertraline, S-citalopram and combinations thereof. SSRIs, or pharmaceutically acceptable salts, polymorphs or stereoisomers thereof, may be combined with NMDA antagonists, such as the aforementioned riluzole, caroverine, memantine and combinations thereof, or pharmaceutically acceptable salts, polymorphs or stereoisomers thereof. Additionally, there are SSRIs with both SSRI and NMDA antagonist activity, such as alaproclate (2-(p-chlorophenyl)-1,1-dimethyl-2-aminopropanoate). The present invention contemplates therapeutic compositions, especially oral compositions, comprising nicergoline, MMDL and/or MDL in combination with one or more of these SSRI/NMDA antagonists, or pharmaceutically acceptable salts, polymorphs or stereoisomers thereof.

a. Fluoxetine

Fluoxetine, or (±)-N-methyl-γ-[4-trifluoromethyl)-phenoxyl benzenepropanamine, is an SSRI known to be an antidepressant.

b. Sertraline

Sertraline, or (1S-cis)4-(3,4-diclorophenyl)-1,2,3,4-tetrahydro-N-methyl-1-naphthalenamine, is an SSRI known to be an antidepressant.

c. S-Citalopram

S-citalopram, or 1-[3-(dimethylamino)propyl]-1-(4-fluorophenyl)-1,3-dihydro-5-isobenzofurancarbonitrile, is an SSIR known to have antidepressant activity. Cruz et al. have presented evidence that suggests that the use of citalopram can have a positive impact on auditory processes in the elderly. Oswaldo Laercio M. Cruz et al, Serotonin Reuptake Inhibitors in Auditory Processing Disorders in Elderly Patients: Preliminary Results, Laryngoscope, 114, September 2004, 1656-1659.

d. Alaproclate

Alaproclate ([2-(p-chlorophenyl)-1,1-dimethyl-2-aminopropanoate]) is a known antidepressant having both selective serotonin reuptake inhibiting and N-methyl-D-aspartate antagonist activity.

e. SSRI and NMDA Antagonist Combinations

Combinations of SSRI and NMDA antagonists include: one or more SSRI selected from the group consisting of fluoxetine, sertraline and S-citalopram with one or more NMDA antagonists selected from the group consisting of riluzole, caroverine, memantine and magnesium. Particular combinations include; fluoxetine and riluzole; fluoxetine and caroverine; fluoxetine and memantine; fluoxetine and magnesium; sertraline and riluzole; sertraline and caroverine; sertraline and memantine; sertraline and magnesium; S-citalopram and riluzole; S-citalopram and caroverine; S-citalopram and memantine; S-citalopram and magnesium; fluoxetine and sertraline with a NMDA antagonist (such as riluzole, caroverine, memantine; magnesium and combinations of two or more thereof) fluoxetine and S-citalopram and a NMDA antagonist (such as riluzole, caroverine, memantine, magnesium and combinations of two or more thereof); sertraline and S-citalopram and a NMDA antagonist (such as riluzole, caroverine, memantine, magnesium and combinations of two or more thereof); fluoxetine, sertraline and S-citalopram and a NMDA antagonist (such as riluzole, caroverine, memantine, magnesium and combinations of two or more thereof). The present invention contemplates therapeutic compositions, especially oral compositions, comprising nicergoline, MMDL and/or MDL in combination with one or more of these combinations of SSRI/NMDA antagonists.

5. Dopamine Releaser/NMDA Antagonists

The present invention contemplates therapeutic compositions, especially oral compositions, comprising nicergoline, MMDL and/or MDL in combination with one or more dopamine releaser/NMDA antagonists, or pharmaceutically acceptable salts, polymorphs or stereoisomers thereof, such as the dopamine releaser/NMDA antagonists described below.

a. Amantadine

Amantadine (tricycle[3.3.1.1^{3,7}]decan-1-amine) has combined dopamine releasing and N-methyl-D-aspartate antagonistic activity. It has been used for the treatment of Parkinsonism, as an antiviral in the treatment of influenza A and for drug-induced extra-pyramidal reactions. In theory, the dopamine releasing effect of amantadine enhances central auditory processing, while the NMDA antagonistic effect protects the inner ear hair cells from glutamate-induced toxicity. Thus, the present invention contemplates therapeutic compositions, especially oral compositions, comprising nicergoline, MMDL and/or MDL in combination with amantadine. It is contemplated that such combinations provide additive or synergistic effects in the treatment or prevention of a hearing disorder.

b. Combinations

A compound having combined dopamine releasing and NMDA antagonistic activity, such as amantadine, can be further combined with one or more additional active ingredients for the prevention or treatment of hearing disorders. Such additional compounds can act in coordination with the dopamine releasing/NMDA antagonist compound. In some embodiments, the additional compounds enhance the hearing disorder protecting or treating activity of the dopamine releaser/NMDA antagonist, allowing treatment at a lower dose of the dopamine releaser/NMDA antagonist, or allowing treatment of patients who would not respond to dopamine releaser/NMDA antagonist therapy at a dose below its toxic dose. In other embodiments, the additional compound or compounds ameliorate one or more undesirable side effects of the dopamine releaser / NMDA antagonist.

In particular embodiments, a compound having combined dopamine releasing and NMDA antagonistic activity, such as amantadine, can be combined with one or more additional active pharmaceutical ingredients, such as one or more member of the group consisting of nicergoline, MMDL or MDL, selective serotonin reuptake inhibitors, and antioxidants. Thus, particular combinations possible within the scope of this invention include: amantadine and a SSRI; amantadine and an antioxidant; amantadine and a SSRI; and amantadine, a SSRI and an antioxidant. Thus, in some embodiments, the composition according to the present invention comprises nicergoline, MMDL and/or MDL in combination with a combination selected from the group consisting of: amantadine and a SSRI; amantadine and an antioxidant; amantadine and a SSRI; and amantadine, a SSRI and an antioxidant. Particular SSRIs and antioxidants are described in more detail above.

6. Acetylcholine Release Inducer/Antioxidant/NMDA Antagonist/NERI

Compounds having combined acetylcholinesterase inducing, antioxidant, N-methyl-D-aspartate antagonist and norepinephrine reuptake inhibiting activity, such as bifemelane, or pharmaceutically acceptable salts, polymorphs or stereoisomers thereof, can be used in the prevention or treatment of hearing disorders, such as hearing loss and tinnitus. In theory, compounds such as bifemelane enhance brain levels of acetylcholine and norepinephrine, thereby improving auditory processing, speech recognition and hearing perception. Also in theory, compounds such as bifemelane, by blocking NMDA receptors and by acting as antioxidants, provide protection to the inner ear hair cells. Thus, the present invention contemplates a therapeutic composition according to the invention comprising a compound having combined acetylcholinesterase inducing, antioxidant, N-methyl-D-aspartate antagonist and norepinephrine reuptake inhibiting activity.

Bifemelane (N-methyl4-[2-(phenylmethyl)phenoxy-1-butanamine) is a compound combining acetylcholinesterase inducing, antioxidant, N-methyl-D-aspartate antagonist and norepinephrine reuptake inhibiting activity that has been used as a nootropic agent. In some embodiments, the invention contemplates a therapeutic composition according to the invention comprising bifemelane and one or more of nicergoline, MMDL or MDL.

7. MAO-A Inhibitor/SRI/Antioxidant

Compounds having combined monamineoxidase-A inhibiting, serotonin reuptake inhibiting and antioxidant activity, or pharmaceutically acceptable salts, polymorphs or stereoisomers thereof, such as pirlindole, can be used alone or in combination with an NMDA antagonist or amantadine for the treatment of a hearing disorder, such as hearing loss or tinnitus. In theory, the central effects of compounds such as pirlindole via increasing norepinephrine and serotonin increase auditory processing, while the antioxidant effect of compounds such as pirlindole protects inner ear hair cells from damage induced by oxidative species. Thus, the present invention contemplates a therapeutic composition according to the invention comprising a compound having combined monamineoxidase-A inhibiting, serotonin reuptake inhibiting and antioxidant activity. In particular embodiments, the present invention contemplates a composition according to the invention comprising pirlindole and one or more of nicergoline, MMDL or MDL.

8. Norepinephrine and Serotonin Reuptake Inhibitor/Low Affinity NMDA Antagonists

Norepinephrine and serotonin reuptake inhibitor/low affinity NMDA antagonists, or pharmaceutically acceptable salts, polymorphs or stereoisomers thereof, such as milnacipran and bicifadine, can be administered along with nicergoline, MMDL or MDL to provide central and peripheral treatment effects. The central effects of such compounds are through the norepinephrine reuptake inhibition and serotonin reuptake inhibition activities, which provide improved auditory processing. The peripheral effects include protection of inner ear hair cells via the N-methyl-D-aspartate antagonist activity. Thus, the present invention contemplates a therapeutic composition according to the invention comprising a compound having combined norepinephrine and serotonin reuptake inhibitor activity, such as milnacipran and bicifadine. In some particular embodiments, the present invention contemplates a composition according to the invention comprising milnacipran and one or more of nicergoline, MMDL or MDL. In other particular embodiments, the present invention contemplates a composition according to the invention comprising bicifadine and one or more of nicergoline, MMDL or MDL.

a. Milnacipran

Milnacipran (cis-(±)-2-(aminomethyl)-N,N-diethyl-1-phenylcyclopropanecarboxamide) is a known antidepressant having norepinephrine reuptake and serotonin reuptake inhibiting effects. Milnacipran is also a weak N-methyl-D-aspartate antagonist. In some embodiments of the invention, nicergoline, MMDL or MDL is used in combination with milnacipran. In particular embodiments, milnacipran and nicergoline, MMDL or MDL are administered in separate dosage forms. In other embodiments, nicergoline, MMDL or MDL and milnacipran are combined in the same dosage form, for example in a common capsule or tablet. In particular embodiments, separate nicergoline, MMDL or MDL and milnacipran dosage forms are combined in a kit, such as a blister pack, as discussed in more detail below.

b. Bicifadine

Bicifadine (1-(4-methylphenyl)-3-azabicyclo[3.1.0]hexane) is a non-opioid analgesic having norepinephrine reuptake inhibiting, serotonin reuptake inhibiting and low affinity NMDA antagonistic activities. In some embodiments of the invention, nicergoline, MMDL or MDL is used in combination with bicifadine. In particular embodiments, bicifadine and nicergoline, MMDL or MDL are administered in separate dosage forms. In other embodiments, nicergoline, MMDL or MDL and bicifadine can be combined in the same dosage form, for example in a common capsule or tablet. In particular embodiments, separate nicergoline, MMDL or MDL and bicifadine dosage forms are combined in a kit, such as a blister pack, as discussed in more detail below.

9. Calcium Channel Antagonists and SSRI or NSRI

In some embodiments, a calcium channel antagonist, or pharmaceutically acceptable salts, polymorphs or stereoisomers thereof, such as nimodipine or verapamil, is used in combination with a selective serotonin reuptake inhibitor (S SRI) or a norepinephrine selective reuptake inhibitor (NSRI) in the prevention or treatment of hearing disorders. Thus, the present invention contemplates a therapeutic composition comprising nicergoline, MMDL and/or MDL in combination with a calcium channel antagonist, such as nimodipine or verapamil, and a selective serotonin reuptake inhibitor or a norepinephrine selective reuptake inhibitor. It is contemplated that such a combination demonstrates additive or synergistic effects in the treatment or prevention of a hearing disorder.

a. Nimodipine

Nimodipine (1,4-dihydro-2,6-dimethyl-4-(3-nitrophenyl)-3,5-pyridinedicarboxylic acid 2-methoxyethyl 1-methoxyethyl ester) is a calcium channel antagonist known to have vasodilating activity.

b. Verapamil

Verapamil (α-[3-[[2-(3,4-dimethoxyphenyl)ethyl]methylamino]propyl]-3,4-dimethoxy-α-(1-methylethyl)-benzeneacetonitrile) is a calcium channel antagonist known to have anti-angina and anti-arrhythmic activity.

c. SSRIs

Selective serotonin reuptake inhibitors, such as fluoxetine, sertraline and S-citalopram, block the reuptake of serotonin in the synaptic cleft, thereby providing greater serotonin-dependent neurotransmission.

d. NSRIs

Norepinephrine selective reuptake inhibitors, such as atomoxetine, selectively block the reuptake of norepinephrine in the synaptic cleft, thereby increasing the functional concentration of this neurotransmitter.

10. 5-HT SRI/NRI/ACh Releaser/NMDA Antagonist

A compound, or a pharmaceutically acceptable salt, polymorph or stereoisomer thereof, having 5-HT serotonin reuptake inhibiting, norepinephrine reuptake inhibiting, acetylcholine releasing and N-methyl-D-aspartate antagonist activity can be used either alone or in addition to an NMDA antagonist for the treatment or prevention of a hearing disorder, such as hearing loss or tinnitus. Thus, the present invention contemplates a therapeutic composition comprising nicergoline, MMDL and/or MDL with an agent having 5-HT serotonin reuptake inhibiting, norepinephrine reuptake inhibiting, acetylcholine releasing and N-methyl-D-aspartate antagonist activity.

a. Indeloxazine

Indeloxazine (2-[(1H-inden-7-yloxy)methyl]morpholine hydrochloride) is known to be an antidepressant, nootropic having 5-HT serotonin reuptake inhibiting, norepinephrine reuptake inhibiting, acetylcholine releasing and N-methyl-D-aspartate antagonist activity. In some embodiments, indeloxazine is used alone for the treatment or prevention of a hearing disorder, such as hearing loss or tinnitus. In theory, the ability of indeloxazine to increase brain serotonin, norepinephrine and acetylcholine improves central auditory processing, speech recognition and hearing perception, while its NMDA blocking activity provides protection to the inner ear hair cells. Thus, the present invention contemplates a therapeutic composition comprising nicergoline, MMDL and/or MDL with indeloxazine.

b. Indeloxazine Plus NMDA Antagonist

In some embodiments, indeloxazine is used together with a compound selected from the group consisting of NMDA antagonists. In theory, the ability of indeloxazine to increase brain serotonin, norepinephrine and acetylcholine improves central auditory processing, speech recognition and hearing perception, while its NMDA blocking activity provides protection to the inner ear hair cells. The additional compound having NMDA antagonist activity provides protection to the inner ear hair cells. NMDA antagonists that may be combined with indeloxazine include riluzole, caroverine, memantine, magnesium and mixtures of two or more thereof. Thus, the present invention contemplates a therapeutic composition comprising nicergoline, MMDL and/or MDL with indeloxazine and a NMDA antagonist. In particular embodiments, the invention contemplates a therapeutic composition according to the invention that comprises nicergoline, MMDL and/or MDL in combination with indeloxazine and an NMDA antagonist selected from riluzole, caroverine, memantine, magnesium and mixtures of two or more thereof.

Particular combinations of indeloxazine and an NMDA antagonist include: indeloxazine and riluzole; indeloxazine and caroverine; indeloxazine and memantine; and indeloxazine and magnesium. Thus, the present invention contemplates compositions according to the present invention comprising nicergoline, MMDL and/or MDL in with a combination of indeloxazine and NMDA antagonist selected from the group consisting of indeloxazine and riluzole; indeloxazine and caroverine; indeloxazine and memantine; and indeloxazine and magnesium.

B. Salts, Stereoisomers, Polymorphs and Derivatives

Although described above with reference specific to compounds, one can also utilize stereoisomers, polymorphs, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington 's Pharmaceutical Sciences, 17th ed. (Mack Publishing Company, Easton, Pa., 1985, p. 1418).

Stereoisomers are compounds made up of the same atoms having the same bond order but having different three-dimensional arrangements of atoms which are not interchangeable. The three-dimensional structures are called configurations. Two kinds of stereoisomers include enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). Diastereomers are two stereoisomers which are not mirror images but also not superimposable. Diastereoisomers have different physical properties and can be separated from one another easily by taking advantage of these differences.

Different polymorphs of the compounds may also be used. Polymorphs are, by definition, crystals of the same molecule having different physical properties as a result of the order of the molecules in the crystal lattice. The polymorphic behavior of drugs can be of crucial importance in pharmacy and pharmacology. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity).

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a patient, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds in vivo. Metabolites include products or intermediates from any metabolic pathway.

C. Formulations

1. General Considerations

The compounds, or pharmaceutically acceptable salts thereof, or polymorphic variations thereof, can be formulated as pharmaceutical compositions. Unless otherwise specified, reference to a base compound, such as nicergoline, MMDL or MDL, encompasses its pharmaceutically acceptable salts and/or polymorphic variations. Such compositions can be administered orally, buccally, intravenously, parenterally, by inhalation spray, rectally, intradermally, transdermally, pulmonary, nasally or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. As specific compounds described herein are considered orally available, it is considered especially advantageous for the composition to be administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach. In addition, combinations of immediate release compositions and delayed release/extended release compositions may be formulated together.

In some embodiments, nicergoline, MMDL or MDL is formulated as the sole active pharmaceutical ingredient (API) in a dosage form. Such nicergoline, MMDL or MDL dosage form may be used alone or in combination therapy with one or more additional dosages containing one or more active pharmaceutical ingredients for prevention or treatment of hearing loss. In such embodiments, the daily dosage of nicergoline, MMDL or MDL is conveniently provided in a single dosage form as described herein, or may be divided amongst two, three, four or more dosages. In some embodiments, the dosage of nicergoline, MMDL or MDL is in the range of about 5 to about 250 mg per day. In particular embodiments, the dosage of nicergoline, MMDL or MDL is in the range of about 10 to 100 mg per day. In specific embodiments, the dosage range for nicergoline, MMDL or MDL is from about 25 to about 50 mg per day.

II. Methods of Use

A. General Administration Protocol

The therapeutic compositions according to the present invention are administered to a patient at a dose effective to protect the mammal from hearing loss, to restore lost hearing or to alleviate one or more symptoms of a hearing disorder, such as decreased speech recognition, tinnitus, vertigo or decreased memory. In some embodiments one of nicergoline, MMDL or MDL is administered as the sole active pharmaceutical ingredient. In other embodiments, two or more of nicergoline, MMDL and MDL are administered as the active pharmaceutical ingredients. In still further embodiments, nicergoline, MMDL and/or MDL are combined with one or more additional active pharmaceutical ingredients. In this context “combined” means using nicergoline, MMDL and/or MDL and at least one additional active pharmaceutical ingredient in the same regime. Thus, nicergoline, MMDL and/or MDL and at least one other active pharmaceutical ingredient can be combined in the same dosage form and thereby administered simultaneously to the mammal being treated. Alternatively, nicergoline, MMDL and/or MDL and at least one other active pharmaceutical ingredient can be compounded (prepared) in separate dosage forms and administered separately, either at substantially the same time (simultaneously) or at substantially different times (sequentially) in a given period of time. For example, nicergoline, MMDL and/or MDL may be administered at bedtime and the other active pharmaceutical ingredient may be administered upon awakening. As another example, nicergoline, MMDL and/or MDL and another active pharmaceutical ingredient may be taken on alternating days. As a further example, nicergoline, MMDL and/or MDL and another active pharmaceutical ingredient may be taken at substantially the same time. In specific cases, at least one additional active pharmaceutical ingredient may be taken at such a time relative to the dosing of nicergoline, MMDL and/or MDL that it counteracts one or more unwanted side effects of nicergoline, MMDL and/or MDL.

In some embodiments, the compositions are administered orally, although other dosing regimens are possible and are not excluded from the invention. In one embodiment, the nicergoline, MMDL and/or MDL and a second active pharmaceutical ingredient are administered simultaneously, either in the same or separate dosage forms. The compositions can be administered as immediate release, sustained release, intermittent release, and/or delayed release formulations. The composition can be administered in a single dose, in escalating doses, or at an initial elevated dose followed by a lower dose after a particular circulating blood concentration of the compound has been achieved. In some embodiments, the drugs are administered in an immediate release twice/day dosing regimen in which the second active pharmaceutical ingredient is given twice daily (BID) in the normal dose range and nicergoline, MMDL or MDL is given twice daily (BID) at half its daily dose.

An intermittent administration protocol may be used where chronic administration is not desirable. In such a regimen, the composition according to the invention is administered in time blocks of several days with a defined minimum washout time between blocks. Intermittent administration occurs over a period of several weeks to months to achieve a significant improvement in the symptoms of hearing disorders.

One of skill in the art should be able to choose administration protocols and determine appropriate dosing regimes to treat symptoms of hearing disorders based on bioavailability and half-life of the compound to be administered. For many of the disclosed compounds, appropriate dosage ranges have been established to maximize circulating concentrations of the compound and minimize side-effects.

The combination of nicergoline, MMDL and/or MDL and with an additional active pharmaceutical ingredient, such as an antioxidant, NMDA antagonist, SSRI or combined SSRI/NMDA antagonist, can be administered for a specific duration to improve symptoms of a particular disorder. A suitable endpoint can be where one symptom of the disorder is treated by administration of the compounds and the treatment considered effective. In other situations, the treatment can be considered effective when more than one symptom is treated.

B. Effective Dosage Ranges

Appropriate dosages can be determined by one of skill in the art based on using routine experimentation and standard techniques utilizing dosages currently approved. Compounds in the disclosed drug classes are known in the art and can be initially administered at similar doses and titrated appropriately to treat symptoms of the disorders and side effects in a given patient. Intra-patient variability is known in the art depending on the severity of symptoms and dosages are commonly adjusted to exact a particular therapeutic effect in a particular patient.

Therapeutically effective amounts for use in humans can also be determined from animal models. For example, a dose for humans can be formulated to achieve a circulating concentration of active pharmaceutical ingredient that has been found to be effective in animals. Effective amounts for use in humans can also be determined from human data for the compounds used to treat other disorders, for example, neurological disorders. The amount administered can be the same amount administered to treat other neurological disorders or can be an amount higher or lower than the amount administered to treat other neurological disorders.

The optimal concentration of the drug in each pharmaceutical formulation varies according to the formulation itself. In some embodiments, the pharmaceutical formulation contains the active pharmaceutical ingredient (API) at a concentration of about 0.1 to 90% by weight (such as about 1-20% or 1-10%). Appropriate dosages of the API can readily be determined by those of ordinary skill in the art of medicine by assessing amelioration of the disorder or side effect in the patient, and increasing the dosage and/or frequency of treatment as desired. The optimal amount of the API may depend upon the mode of administration, the age and the body weight of the patient, and the condition of the patient. In some embodiments, the API is administered at a dosage of 0.1 to 1.0 mg/kg. In some embodiments, the daily dose of nicergoline in humans is in the range of about 0.5 to 200 mg per day, especially about 1 to about 100 mg per day, and more particularly about 5 to about 60 mg per day. It is to be understood that dosage of MMDL and MDL will vary from that of nicergoline based on their oral or parenteral bioavailability, their pharmacokinetic properties, etc. In general, the dose of MMDL and MDL will be from about 5% to about 500%, especially about 50% to about 250% of the equivalent (on a molar basis) daily dose of nicergoline. It is to be understood that for veterinary use, the daily dose will be scaled to the size of the particular animal to be treated. Preferred daily doses of the antioxidants, NMDA antagonists, SSRI or combined SSRI/NMDA antagonists are approximately 1 to 500 mg/day, and more advantageously 4 to 250 mg/day.

C. Treatment Strategies

The invention provides methods of treating or preventing a hearing disorder. In some embodiments, the invention provides a method of protecting against noise-induced hearing loss, treating tinnitus, reducing transmission of abnormal sounds and auditory sensations associated with tinnitus, reducing fluid accumulation in the inner ear, facilitating central auditory processing of sounds and speech, or combinations thereof, comprising administering nicergoline, MMDL and/or MDL to a mammal in need of such treatment in an amount sufficient to protect against noise-induced hearing loss, reduce transmission of abnormal sounds and auditory sensations associated with tinnitus, reduce fluid accumulation in the inner ear and/or facilitate central auditory processing of sounds and speech. Unless otherwise specified, treatment of one or more of the above hearing disorders is not exclusive of treatment of one or more additional hearing disorders. Moreover, treatment of a hearing disorder is not exclusive of prevention of the same or another hearing disorder, nor is prevention of a hearing disorder exclusive of treatment of the same or another hearing disorder, unless otherwise specified.

In some advantageous embodiments, the invention comprises administering a therapeutic amount of a therapeutic composition comprising MDL to a mammal in need of such treatment. In some embodiments, the therapeutic composition consists essentially of MDL. In other embodiments, the therapeutic composition comprises one or more additional active ingredients other than nicergoline and MMDL. In still further embodiments, the therapeutic composition further comprises one or more additional active ingredients, such as antioxidants or spin trapping agents, norepinephrine reuptake inhibitors, epinephrine reuptake inhibitors, norepinephrine-epinephrine reuptake inhibitors N-methyl-D-aspartate antagonist, a compound that blocks the excitotoxic actions of glutamate within the inner ear, thereby blocking the glutamate-mediated noise-induced damage to the hair cells of the cochlea of the inner ear.

In particular embodiments, the invention provides methods of protecting against noise-induced damage or loss of hair cells in the inner cochlea of the inner ear. The method comprises administering nicergoline, MMDL and/or MDL alone or in combination with one or more additional active ingredients to a mammal in need of protection from noise-induced hearing loss in an amount sufficient to protect against noise-induced hearing loss. In some embodiments, nicergoline, MMDL and/or MDL is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of reducing transmission of abnormal sounds and auditory sensations associated with tinnitus. The method comprises administering nicergoline, MMDL and/or MDL alone or in combination with one or more additional active ingredients to a mammal in need of reducing transmission of abnormal sounds and auditory sensations associated with tinnitus in an amount sufficient to reduce transmission of abnormal sounds and auditory sensations associated with tinnitus. In some embodiments, nicergoline, MMDL and/or MDL is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of reducing fluid accumulation associated with trauma and other disorders of the inner ear. The method comprises administering nicergoline, MMDL and/or MDL alone or in combination with one or more additional active ingredients to a mammal in need of reducing fluid accumulation associated with trauma and other disorders of the inner ear in an amount sufficient to reduce fluid accumulation. In some embodiments, nicergoline, MMDL and/or MDL is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of stimulating central nervous system serotonin neurotransmission, thereby facilitating central auditory processing of sounds and speech. The method comprises administering nicergoline, MMDL and/or MDL alone or in combination with one or more additional active ingredients to a mammal in need of such treatment in an amount sufficient to facilitate central auditory processing. In some embodiments, nicergoline, MMDL and/or MDL is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

Thus, the invention provides both methods of protecting against hearing loss and methods of treating hearing loss. In this context, protecting against hearing loss means that the active pharmaceutical ingredient or ingredients protect, at least to some degree, against the loss of hearing in a mammal. Such protection may range from slight to nearly complete. The mammal treated may be one that has already experienced hearing loss, including one that has already experienced hearing loss and is expected to be subjected to conditions similar to those that brought about the current degree of hearing loss. The mammal treated may also be one that has yet to experience notable hearing loss but is expected to be at risk for hearing loss, due to genetic profiling, expected exposure to one or more hearing-loss inducing causes (such as excessive noise), or a combination of those factors.

In the context of the methods according to the invention, treatment of hearing loss means restoring (at least in part) hearing to the mammal, or ameliorating one or more symptoms of hearing loss. Symptoms of hearing loss include experiencing abnormal sounds and auditory sensations associated with tinnitus and reduced ability to distinguish sounds and/or spoken words. Thus, for the purposes of this invention, hearing disorders include noise-induced hearing loss, drug-induced hearing loss, drug-induced tinnitus, and central auditory processing disorder (CAPD).

The treated mammal (patient) is a human or a non-human mammal, such as a dog, a cat, a monkey, an ape, a gerbil, a hamster, a mouse, a rat, a horse, a cow, a rabbit or other mammal. It is expected that, while the dosing and other considerations may change from species to species, the person of skill in the art will be able to adapt the disclosed nicergoline, MMDL and/or MDL treatment regimes to treat a variety of mammalian species that are currently experiencing hearing loss or are expected to face the threat of experiencing hearing loss.

D. Treatment of Particular Disorders

Methods of the invention can be used to treat a variety of hearing disorders, including noise-induced hearing loss; drug-induced hearing disorders; central auditory processing disorders; and other hearing-related disorders as described in more detail below.

1. Treatment of Noise-Induced Hearing Loss

In particular embodiments, the invention provides methods of protecting against noise-induced damage or loss of hair cells in the inner cochlea of the inner ear. The method comprises administering nicergoline, MMDL and/or MDL alone or in combination with one or more additional active ingredients to a mammal in need of protection from noise-induced hearing loss in an amount sufficient to protect against noise-induced hearing loss. In some embodiments, nicergoline, MMDL and/or MDL is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

2. Treatment And Prevention of Drug-Induced Hearing Disorders

In some embodiments, the invention provides methods of treating or preventing one or more drug-induced hearing disorders. The method comprises administering to a patient a therapeutically effective amount of a composition comprising nicergoline, MMDL and/or MDL alone or in combination with one or more additional active. In some embodiments, the composition comprising nicergoline, MMDL and/or MDL further comprises at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, the composition comprising nicergoline, MMDL and/or MDL is administered to the patient in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In some embodiments, the invention provides a method for preventing a drug-induced hearing disorder, such as hearing loss or tinnitus. In some such embodiments, the invention provides a method for preventing the onset of a drug-induced hearing disorder. In particular, the invention provides methods for preventing a drug-induced hearing disorder, comprising administering a therapeutically effective amount of composition comprising nicergoline, MMDL and/or MDL to a patient prior to administering to that patient one or more chemotherapeutic agents for the treatment of a condition other than a hearing disorder. In some embodiments, treatment with the composition comprising nicergoline, MMDL and/or MDL can begin substantially before treatment with the other chemotherapeutic agent. (For purposes of this invention, “other chemotherapeutic agent” includes antineoplastic and antibacterial compounds as described in more detail below.) In exemplary embodiments, treatment with a therapeutic composition comprising nicergoline, MMDL and/or MDL can begin up to one month prior to treatment with the other chemotherapeutic agent, although prophylactic pre-treatment can vary from about 1 day to about 60 days, depending upon the particular chemotherapy contemplated.

In still further embodiments, the invention provides a method of preventing ototoxic effects of one or more chemotherapeutic agents, which includes concurrent dosing of a therapeutic composition comprising nicergoline, MMDL and/or MDL with the other chemotherapeutic agent. (Such ototoxic effects include drug-induced hearing loss and tinnitus.) In some embodiments, dosing of the therapeutic composition comprising nicergoline, MMDL and/or MDL begins on the same day as dosing of the other chemotherapeutic agent. Treatment with a composition comprising nicergoline, MMDL and/or MDL can then be continued for the duration of chemotherapeutic agent, or may continue for some time, e.g. from about 1 to about 90 days after cessation of the chemotherapeutic agent. In some cases, it will be sufficient to continue administration of the therapeutic composition comprising nicergoline, MMDL and/or MDL treatment for a time after cessation of administration of the other chemotherapeutic agent that is equivalent to the washout period (plus or minus a few days) for the other chemotherapeutic agent. The washout period will vary based on the rate of clearance of the chemotherapeutic agent from the body, and can be determined by one of skill in the art by art-recognized methods.

In other embodiments, the invention provides a method for treating a drug-induced hearing disorder, such as drug-induced hearing loss or tinnitus. Such treatment can include amelioration of drug-induced hearing loss, reduction or elimination of tinnitus, partial or total rehabilitation of hearing, or prevention of further hearing loss arising out of ototoxic effects of chemotherapeutic agents. In such embodiments, the invention provides for dosing of a therapeutic composition comprising nicergoline, MMDL and/or MDL in response to a noted decrease in hearing function arising out of, or occurring during, dosing of one or more chemotherapeutic agents. Dosing of a therapeutic composition comprising nicergoline, MMDL and/or MDL can then continue for the duration of chemotherapeutic treatment, and for at least one day after cessation of chemotherapeutic treatment. In some embodiments, dosing of nicergoline therapeutic composition comprising nicergoline, MMDL and/or MDL can continue for at least a washout period for the chemotherapeutic agent after the last dose of chemotherapeutic agent has be administered to the patient. In particular embodiments, dosing of nicergoline, MMDL and/or MDL can persist for about 1 to 90 days after cessation of chemotherapy. In other embodiments, dosing of nicergoline, MMDL and/or MDL can persist indefinitely, for example until the clinician is satisfied that the danger of ototoxic hearing loss has passed or until the clinician determines that drug-induced hearing loss and/or tinnitus has been ameliorated to a desired degree.

A number of drugs have been found to elicit ototoxic effects in at least some patients. Embodiments of the invention comprise treatment of ototoxic sided effects, such as hearing loss and tinnitus, in a patient has been, is being or will be treated with one or more ototoxic drugs. Some examples of ototoxic drugs include certain antibacterial and antineoplastic drugs. For example, some ototoxic drugs contemplated within the scope of the present invention are chemotherapeutic agents, e.g. antineoplastic agents, and antibiotics. Other possible candidates include loop-diuretics, quinines or a quinine-like compound, and salicylate or salicylate-like compounds. Thus, the present invention provides a method for treating or preventing a drug-induced hearing disorder caused by an ototoxic agent, wherein the ototoxic agent is an antineoplastic agent such as cisplatin, an antibiotic such as an aminoglycoside, a loop-diuretic, a quinine, a quinine-like compound, a salicylate or salicylate-like compound. The method comprises administering to a patient a therapeutically effective amount of a therapeutic composition comprising nicergoline, MMDL and/or MDL, wherein the therapeutically effective amount is an amount sufficient to treat or prevent a drug-induced hearing disorder, such as drug-induced hearing loss and/or tinnitus.

In particular embodiments, the invention provides methods for treating or preventing a drug-induced hearing disorder, such as drug-induced hearing loss or tinnitus, wherein the ototoxic agent is an antineoplastic agent such as cisplatin, an antibiotic such as an aminoglycoside (as described below), a loop-diuretic, a quinine, a quinine-like compound, a salicylate or salicylate-like compound. The method comprises administering to a patient a therapeutically effective amount of a therapeutic composition comprising amount of nicergoline, MMDL and/or MDL. The drug-induced hearing disorder may include drug-induced hearing loss, tinnitus or both.

Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2. Thus, embodiments of the present invention provide methods of treating or preventing a drug-induced hearing disorder, such as drug-induced hearing loss or tinnitus, comprising administering to a patient, who has been, is being or will be treated with one or more aminoglycosides, a therapeutically effective amount of a composition comprising nicergoline, MMDL and/or MDL.

Hearing impairments induced by aminoglycosides can be prevented or reduced by the methods of the invention. Although the aminoglycosides are particularly useful due to their rapid bactericidal action against infections of aminoglycoside-susceptible organisms, their use has heretofore been limited to more severe, complicated infections because of ototoxic and nephrotoxic side-effects. For this reason the aminoglycosides have been considered to have a low therapeutic/risk ratio compared to other antibiotics used systemically. Thus, the invention provides improved methods of treatment of aminoglycoside-susceptible infections, comprising administering to a patient an ant bacterially effective amount of an aminoglycoside and a therapeutic composition comprising nicergoline, MMDL and/or MDL. It is to be recognized that therapeutic doses of aminoglycosides have been established; and the present invention contemplates administration of aminoglycosides in a range of about 100 to about 500%, in particular about 100 to about 250%, and more particularly about 100 to about 150% of the currently recommended doses, which are available in general in the product labeling and package inserts for the commercially available drug aminoglycoside drug products. The improved methods provide prophylaxis against aminoglycoside-induced hearing loss and/or tinnitus, thereby expanding the therapeutic index of the aminoglycoside drug.

In some embodiments the composition of the invention is co-administered with an ototoxin in the same dosage form. For example, an improved method is provided for treatment of infection of a mammal by administration of an aminoglycoside antibiotic, the improvement comprising administering a therapeutically effective amount of a composition comprising nicergoline, MMDL and/or MDL and an antibiotic.

In other embodiments, the aminoglycoside antibiotic and the therapeutic composition comprising nicergoline, MMDL and/or MDL are administered to the patient in separate dosage forms.

In yet another embodiment is provided an improved method for treatment of cancer in a mammal by administration of a chemotherapeutic compound; the improvement comprising administering to a patient a therapeutically effective amount of a composition comprising nicergoline, MMDL and/or MDL and an antineoplastic agent.

In other embodiments, the antineoplastic agent and the therapeutic composition comprising nicergoline, MMDL and/or MDL are administered to the patient in separate dosage forms.

Ototoxic antineoplastic chemotherapeutic agents include cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas. Thus, the methods of the invention may be used to treat ototoxicity, (such as drug-induced hearing loss), in a patient that will be, is being, or has been treated with an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas.

3. Treatment of Central Auditory Processing Disorder

Central auditory processing (CAP) relates to the efficiency and efficacy with which the central nervous system (CNS) utilizes auditory information. More specifically, CAP relates to the perceptual processing of auditory information in the CNS as well as the neurobiological activity that underlies that processing and gives rise to electro-physiological auditory potentials. CAP includes the auditory mechanisms that underlie the following abilities or skills: sound localization and lateralization; auditory discrimination; auditory pattern recognition; temporal aspects of audition, including temporal integration, temporal discrimination (e.g. temporal gap detection), temporal ordering and temporal masking; auditory performance in competing acoustic signals (including dichotic listening) and auditory performance with degraded acoustic signals. Central auditory processing disorders, then, relate to difficulties in the perceptual processing of auditory information in the CNS as demonstrated by poor performance in one or more of the above skills. Central auditory processing disorders (CAPD). In some embodiments of the invention, there are provided methods for treatment of CAPD, comprising administering to a patient a therapeutically effective amount of a composition comprising one or more of nicergoline, MMDL or MDL.

Tests for CAPD are known in the art. See generally, American Speech-Language Hearing Association, (Central) Auditory Processing Disorders, 1-20, (2005), available at http://www.asha.org/members/deskref-journals/deslref/default. Suitable tests available for central auditory assessment include: auditory discrimination tests; auditory temporal processing and patterning tests; dichotic speech tests; monaural low-redundancy speech tests; binaural interaction tests; electroacoustic measures; and electrophysiologic measures. Such methods are known to known audiologists and are described generally in American Speech-Language Hearing Association, (Central) Auditory Processing Disorders, 6-7.

In other embodiments, the invention provides methods of reducing transmission of abnormal sounds and auditory sensations associated with tinnitus. The method comprises administering nicergoline, MMDL and/or MDL alone or in combination with one or more additional active ingredients to a mammal in need of reducing transmission of abnormal sounds and auditory sensations associated with tinnitus in an amount sufficient to reduce transmission of abnormal sounds and auditory sensations associated with tinnitus. In some embodiments, nicergoline, MMDL and/or MDL is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

4. Prevention or Treatment of Other Hearing-Related Disorders

In other embodiments, the invention provides methods of reducing fluid accumulation associated with trauma and other disorders of the inner ear. The method comprises administering to a patient a therapeutic composition comprising nicergoline, MMDL and/or MDL alone or in combination with one or more additional active ingredients. In general, the patient will be a mammal in need of reducing fluid accumulation associated with trauma and other disorders of the inner ear. The therapeutically effective amount will be an amount sufficient to reduce fluid accumulation. In some embodiments, the therapeutic composition comprises nicergoline, MMDL and/or MDL and at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, the therapeutic composition comprising nicergoline, MMDL and/or MDL is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of facilitating central auditory processing of sounds and speech by stimulating the intrasynaptic concentration of 5-HT. The method comprises administering to a patient a therapeutic composition comprising nicergoline, MMDL and/or MDL alone or in combination with one or more additional active ingredients. The patient will be a mammal in need of such treatment. The therapeutically effective amount will be an amount sufficient to facilitate central auditory processing. In some embodiments, the therapeutic composition comprising nicergoline, MMDL and/or MDL will be combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, therapeutic composition comprising nicergoline, MMDL and/or MDL will be administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

Thus, the invention provides both methods of protecting against hearing loss and methods of treating hearing loss. In this context, protecting against hearing loss means that the active pharmaceutical ingredient or ingredients protect, at least to some degree, against the loss of hearing in a mammal. Such protection may range from slight to nearly complete. The mammal treated may be one that has already experienced hearing loss, including one that has already experienced hearing loss and is expected to be subjected to conditions similar to those that brought about the current degree of hearing loss. The mammal treated may also be one that has yet to experience notable hearing loss but is expected to be at risk for hearing loss, due to genetic profiling, expected exposure to one or more hearing-loss inducing causes (such as excessive noise), or a combination of those factors.

In the context of the methods according to the invention, treatment of hearing loss means restoring (at least in part) hearing to the mammal, or ameliorating one or more symptoms of hearing loss. Symptoms of hearing loss include experiencing abnormal sounds and auditory sensations associated with tinnitus and reduced ability to distinguish sounds and/or spoken words.

The treated mammal may be human or a non-human mammal such as a dog, a cat, a monkey, an ape, a gerbil, a hamster, a mouse, a rat, a horse, a cow, a rabbit or other mammal. It is expected that, while the dosing and other considerations may change from species to species, the person of skill in the art will be able to adapt the disclosed nicergoline, MMDL and/or MDL treatment regimes to treat a variety of mammalian species that are currently experiencing hearing loss or are expected to face the threat of experiencing hearing loss.

E. Treatment with Specific Compositions

The methods of the invention can be affected with a variety of therapeutic compositions as described in more detail below.

1. Nicergoline, MMDL and/or MDL as API

The invention provides methods of preventing or treating a hearing disorder with nicergoline, MMDL and/or MDL. In some particular embodiments, the invention provides methods of preventing or treating a hearing disorder with nicergoline as the sole active pharmaceutical ingredient. Such method provides for administration to a patient a therapeutically effective amount of nicergoline as the sole active pharmaceutical ingredient. In other particular embodiments, the invention provides methods of preventing or treating a hearing disorder with MMDL as the sole active pharmaceutical ingredient. Such method provides administering to a patient a therapeutic amount of MMDL as the sole active pharmaceutical ingredient. In still further embodiments, the invention provides methods of preventing or treating a hearing disorder with MDL as the sole active pharmaceutical ingredient. Such method provides for administration to a patient a therapeutically effective amount of MDL as the sole active pharmaceutical ingredient.

The method according to the invention includes administering to a patient a therapeutic amount of nicergoline, MMDL and/or MDL. In particular, the invention provides methods of providing protection against noise-induced loss and damage to hair cells within the cochlea of the inner ear, comprising administering a therapeutic amount of nicergoline, MMDL and/or MDL to a patient in need of such treatment.

In particular embodiments, the invention provides methods of protecting against noise-induced hearing loss, reducing transmission of abnormal sounds and auditory sensations associated with tinnitus, reducing fluid accumulation in the inner ear and/or facilitating central auditory processing of sounds and speech, comprising administering to a mammal in need of such treatment an amount of nicergoline, MMDL and/or MDL sufficient to protect against noise-induced hearing loss, reducing transmission of abnormal sounds and auditory sensations associated with tinnitus, reducing fluid accumulation in the inner ear and/or facilitating central auditory processing of sounds and speech.

As mentioned above, several mutant alleles of CYP2D6, also known as dextromethorphan O-demethylase, have been identified. In particular, individuals having normal CYP2D6 function who have been dosed with nicergoline tend to have almost no detectable nicergoline in blood plasma, and very little MMDL as well, the vast majority of nicergoline being first converted to MMDL, and the vast majority of MMDL then being converted to MDL by CYP2D6. In contrast, poor metabolizers, i.e. those who express mutant alleles of CYP2D6 that have low N1-demethylation activity, tend to have relatively high concentrations of MMDL and concomitantly low concentration of MDL in plasma. It is considered that poor metabolizers also tend to represent a subclass of patient who respond poorly to nicergoline therapy. Thus, it is considered that direct administration of MDL, circumventing the CYP2D6-mediated mechanism for N1-demethylation of nicergoline, will improve patient response to therapy directed at treating or preventing hearing disorders. In particular cases, the practitioner may first determine whether the patient expresses a mutant allele of the CYP2D6 gene, and if so, administer to the patient MDL instead of, or in addition to, nicergoline and/or MMDL. In other embodiments, the practitioner may administer MDL to the patient instead of, or in addition to, nicergoline and/or MMDL, thereby avoiding the CYP2D6 mechanism altogether, and thereby reducing the clinical variability in response to therapy associated with polymorphism in the CYP2D6 gene.

Thus, in some embodiments, the invention provides a method of treating or preventing a hearing disorder, comprising administering to a patient known to express a mutant allele of the CYP2D6 gene a therapeutically effective amount of a therapeutic composition comprising MDL, or a pharmaceutically acceptable salt or polymorph thereof. Such a therapeutic composition may also contain one or more additional active and/or inactive ingredients as further described herein. Additionally, or alternatively, the therapeutic composition comprising MDL may be co-administered with one or more additional active ingredients in a separate dosage form.

In other embodiments, the invention provides a method of reducing the variability in treatment or prevention of a hearing disorder, comprising administering to a patient a therapeutically effective amount of a therapeutic composition comprising MDL, or a pharmaceutically acceptable salt or polymorph thereof. The method is especially suitable for treating a population of patients, an unknown sized sub-population of which may express a mutant allele of CYP2D6. In such embodiments, testing for presence or absence of the CYP2D6 mutation is unnecessary and may be eliminated, because the method provides to each treated patient the active metabolite of nicergoline. Such a therapeutic composition may also contain one or more additional active and/or inactive ingredients as further described herein. Additionally, or alternatively, the therapeutic composition comprising MDL may be co-administered with one or more additional active ingredients in a separate dosage form.

2. Nicergoline, MMDL or MDL and Antioxidants or Spin Trapping Agents

In some embodiments, the invention provides a method of protecting against noise-induced hearing loss, reducing transmission of abnormal sounds and auditory sensations associated with tinnitus, reducing fluid accumulation in the inner ear, facilitating central auditory processing of sounds and speech, or combinations thereof, comprising administering nicergoline, MMDL and/or MDL and one or more active pharmaceutical agents that bind to or metabolize reactive oxygen species and provide protection against the damage induced by oxygen species, which are toxic mediators. In some such embodiments, the nicergoline, MMDL and/or MDL is administered in combination with an antioxidant or spin trapping agent. In some such embodiments, nicergoline, MMDL and/or MDL is administered in combination with allopurinol, methionine or L-camitine. In particular embodiments, nicergoline, MMDL and/or MDL is administered in combination with allopurinol. In other particular embodiments, nicergoline, MMDL and/or MDL is administered in combination with glutathione. In still further particular embodiments, nicergoline, MMDL and/or MDL is administered in combination with methionine. In yet further embodiments, nicergoline, MMDL and/or MDL is administered in combination with L-camitine. In yet further embodiments, nicergoline, MMDL and/or MDL is administered in combination with two or more antioxidants, such as allopurinol, glutathione, methionine, or L-carnitine. In still further embodiments, nicergoline, MMDL and/or MDL is administered in combination with one or more antioxidants, such as allopurinol, glutathione, methionine, or L-camitine, and one or more other active pharmaceutical ingredients, such as one or more NMDA antagonists, one or more SSRIs or one or more compounds having both SSRI and NMDA antagonist activity, such as alaproclate (2-(p-chlorophenyl)-1,1-dimethyl 2-aminopropanoate).

In some embodiments, nicergoline, MMDL and/or MDL is administered in the same dosage form as one or more antioxidants or spin trapping agents. In some such embodiments, the nicergoline, MMDL and/or MDL is mixed with one or more antioxidants or spin trapping agents. In others, the nicergoline, MMDL and/or MDL is segregate from the antioxidant or spin trapping agent by a coating, a shell, a capsule or some other means for preventing admixture of nicergoline, MMDL and/or MDL with the antioxidant or spin trapping agent, while maintaining both ingredients in the same dosage form.

3. Nicergoline, MMDL and/or MDL and NMDA Antagonists

In some embodiments, the invention provides a method of protecting against noise-induced hearing loss, reducing transmission of abnormal sounds and auditory sensations associated with tinnitus, reducing fluid accumulation in the inner ear, facilitating central auditory processing of sounds and speech, or combinations thereof, comprising administering nicergoline, MMDL and/or MDL and one or more active pharmaceutical agents that block the excitotoxic actions of glutamate within the inner ear. Glutamate is a mediator of noise-induced damage to the hair cells of the inner ear and blocking N-methyl-D-aspartate (NMDA) receptors provides protection against the toxic effects of glutamate. In some embodiments, nicergoline, MMDL and/or MDL is administered in a single dosage form comprising nicergoline, MMDL and/or MDL and a NMDA antagonist.

In some such embodiments, nicergoline, MMDL and/or MDL is administered in a single dosage form comprising an antagonist of N-methyl-D-aspartate, such as magnesium, riluzole, caroverine, memantine or a combination of two or more thereof. In particular embodiments, nicergoline, MMDL and/or MDL is administered in a single dosage form comprising riluzole. In other particular embodiments, nicergoline, MMDL and/or MDL is administered in a single dosage form comprising caroverine. In still further particular embodiments, nicergoline, MMDL and/or MDL is administered in a single dosage form comprising memantine. In still further particular embodiments, nicergoline, MMDL and/or MDL is administered in a single dosage form comprising magnesium. In yet further embodiments, nicergoline, MMDL and/or MDL is administered in a dosage form comprising two or more NMDA antagonists, such as magnesium, riluzole, caroverine, or memantine. In still further embodiments, nicergoline, MMDL and/or MDL is administered in a single dosage form comprising one or more NMDA antagonists, such as magnesium, riluzole, caroverine, or memantine, and one or more other active pharmaceutical ingredients, such as one or more antioxidants or spin trapping agents, one or more SSRIs or one or more compounds having both SSRI and NMDA antagonist activity.

In some such embodiments, nicergoline, MMDL and/or MDL is administered in a dosage form separate from that contains an NMDA antagonist, such as magnesium, riluzole, caroverine, memantine or a combination of two or more thereof. In particular embodiments, nicergoline, MMDL and/or MDL is administered in one dose and magnesium is administered in a separate dose. In particular embodiments, nicergoline, MMDL and/or MDL is administered in one dose and riluzole is administered in a separate dose. In other particular embodiments, nicergoline, MMDL and/or MDL is administered in one dose and caroverine is administered in a separate dose. In still further particular embodiments, nicergoline, MMDL and/or MDL is administered in one dose and memantine is administered in another dose. In yet further embodiments, nicergoline, MMDL and/or MDL is administered in one dose and two or more NMDA antagonists, such as magnesium, riluzole, caroverine, or memantine are administered in a separate dose. In still further embodiments, nicergoline, MMDL and/or MDL is administered one dose and one or more NMDA antagonists, such as magnesium, riluzole, caroverine, or memantine, and one or more other active pharmaceutical ingredients, such as one or more antioxidants or spin trapping agents, one or more SSRIs or one or more compounds having both SSRI and NMDA antagonist activity, are administered in a separate dose.

As mentioned above, in some embodiments, nicergoline, MMDL and/or MDL is administered in the same dosage form as one or more NMDA antagonists. In some such embodiments, the nicergoline, MMDL and/or MDL is mixed directly with one or more NMDA antagonists. In other embodiments, the nicergoline, MMDL and/or MDL is segregate from one or more NMDA antagonists by a coating, a shell, a capsule or some other means for preventing admixture of nicergoline, MMDL and/or MDL with the antioxidant or spin trapping agent, while maintaining both ingredients in the same dosage form.

4. Nicergoline, MMDL and/or MDL and SSRI/NMDA Antagonists

In some embodiments, the invention provides a method of protecting against noise-induced hearing loss, reducing transmission of abnormal sounds and auditory sensations associated with tinnitus, reducing fluid accumulation in the inner ear, facilitating central auditory processing of sounds and speech, or combinations thereof, comprising administering to a patient nicergoline, MMDL and/or MDL and one or more active pharmaceutical agents that enhance synaptic levels of serotonin in the brain and enhance hearing by improving auditory processing, increasing the signal: noise ratio of environmental sounds, and/or by heightening attention.

In some such embodiments, nicergoline, MMDL and/or MDL is administered in combination with a selective serotonin reuptake inhibitor (SSRI). In some such embodiments, nicergoline, MMDL and/or MDL is administered in combination with fluoxetine, sertraline, S-citalopram or combinations thereof. In particular embodiments, nicergoline, MMDL and/or MDL is administered in combination with fluoxetine. In other particular embodiments, nicergoline, MMDL and/or MDL is administered in combination with sertraline. In still further particular embodiments, nicergoline, MMDL and/or MDL is administered in combination with S-citalopram. In still further embodiments, nicergoline, MMDL and/or MDL is administered in combination with two or more SSRI agents, such as fluoxetine, sertraline or S-citalopram.

In some advantageous embodiments, nicergoline, MMDL and/or MDL is administered in combination with at least one SSRI and at least one NMDA antagonist. In exemplary embodiments, nicergoline, MMDL and/or MDL is administered in combination with at least one SSRI (such as fluoxetine, sertraline, S-citalopram or combinations of two or more thereof) and one or more NMDA antagonists selected from magnesium, riluzole, caroverine and memantine. In some particular embodiments, nicergoline, MMDL and/or MDL is administered in combination with at least one SSRI (such as fluoxetine, sertraline, S-citalopram or combinations of two or more thereof) and magnesium. In some particular embodiments, nicergoline, MMDL and/or MDL is administered in combination with at least one SSRI (such as fluoxetine, sertraline, S-citalopram or combinations of two or more thereof) and riluzole. In other particular embodiments, nicergoline, MMDL and/or MDL is administered in combination with at least one SSRI (such as fluoxetine, sertraline, S-citalopram or combinations of two or more thereof) and caroverine. In yet other embodiments, nicergoline, MMDL and/or MDL is administered in combination with at least one SSRI (such as fluoxetine, sertraline, S-citalopram or combinations of two or more thereof) and memantine. In yet further embodiments, nicergoline, MMDL and/or MDL is administered in combination with at least one SSRI (such as fluoxetine, sertraline, S-citalopram or combinations of two or more thereof) and a combination of two or more of magnesium, riluzole, caroverine and memantine.

In some embodiments, nicergoline, MMDL and/or MDL is administered in a combination comprising at least one at least one agent having combined SSRI and NMDA antagonist activity.

In some embodiments, nicergoline, MMDL and/or MDL is administered in a dosage form comprising at least one agent having both SSRI and NMDA antagonist activity or at least one SSRI and at least one NMDA antagonist. In some such embodiments, the dosage form further comprises at least one antioxidant or spin trapping agent.

In some embodiments, nicergoline, MMDL and/or MDL is administered in a dosage form separate from at least one agent having both SSRI and NMDA antagonist activity or nicergoline, MMDL and/or MDL is administered in a dosage form separate from at least one SSRI or at least one NMDA antagonist. In some such embodiments, the nicergoline, MMDL and/or MDL is mixed with one or more at least one agent having both SSRI and NMDA antagonist activity or nicergoline, MMDL and/or MDL is mixed with at least one SSRI or at least one NMDA antagonist. In other embodiments, the nicergoline, MMDL and/or MDL is segregate from the SSRI NMDA antagonist, SSRI or NMDA antagonist by a coating, a shell, a capsule or some other means for preventing admixture of nicergoline, MMDL and/or MDL and the other active pharmaceutical ingredient, while maintaining the ingredients in the same dosage form.

5. Amantadine and Nicergoline, MMDL and/or MDL

The invention provides methods of preventing or treating a hearing disorder in a mammal, such as a human, using amantadine. Amantadine is a dopamine releaser and a N-methyl-D-aspartate antagonist. The dopamine releasing effect of amantadine will enhance auditory processing, while the NMDA antagonistic effect will protect inner ear hair cells from glutamate-induced toxicity. Nicergoline's, MMDL's and/or MDL's calcium channel antagonist activity will provide protection against noise-induced loss and damage of hair cells within the cochlea of the inner ear. Nicergoline, MMDL and/or MDL's antioxidant activity will reduce the toxic effects of oxidant and free radical species in the peripheral auditory system. Nicergoline, MMDL and/or MDL's glutamate reuptake stimulator activity will provide protection against the toxic effects of glutamate. Thus, the invention provides a method of preventing or treating hearing loss, an auditory disorder such as tinnitus, or both in a mammal, such as a human, comprising administering a therapeutic amount of a combination of amantadine and nicergoline, MMDL and/or MDL to the mammal.

In some embodiments, the method comprises administering to the mammal amantadine and nicergoline, MMDL and/or MDL combined in the same dosage form. In particular embodiments, amantadine and nicergoline, MMDL and/or MDL mixed together. In other embodiments, amantadine and nicergoline, MMDL and/or MDL are combined with one or more excipients to form a biphasic dosage form, wherein amantadine and nicergoline, MMDL and/or MDL occupy separate phases.

In other embodiments, amantadine and nicergoline, MMDL and/or MDL are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of amantadine and nicergoline, MMDL and/or MDL are provided in a kit, such as is defined in more detail below. In specific embodiments, the separate dosages are provided in a kit including instructions for the administration of amantadine and nicergoline, MMDL and/or MDL for the prevention or treatment of hearing disorders, especially for the prevention or treatment of hearing loss, tinnitus or both.

6. Bifemelane and Nicergoline, MMDL and/or MDL

The invention provides methods of preventing or treating a hearing disorder in a mammal, such as a human, using bifemelane in combination with nicergoline, MMDL and/or MDL. Bifemelane is an acetylcholine release inducer, an antioxidant, a N-methyl-D-aspartate antagonist and a norepinephrine reuptake inhibitor. The ability of bifemelane to enhance brain levels of acetylcholine and norepinephrine improves auditory processing, speech recognition and hearing perception. The ability of bifemelane to block N-methyl-D-aspartate receptors and to act as an antioxidant provides protection to the inner ear cells. Thus, the invention provides a method of preventing or treating hearing loss, an auditory disorder, such as tinnitus, or both in a mammal, such as a human. The method comprises administering a therapeutic amount of a combination of bifemelane and nicergoline, MMDL and/or MDL to the mammal.

7. Pirlindole and Nicergoline, MMDL and/or MDL

The invention provides methods of preventing or treating a hearing disorder in a mammal, such as a human, using pirlindole, which is a monamineoxidase-A inhibitor, a serotonin reuptake inhibitor and an antioxidant in combination with nicergoline, MMDL and/or MDL. Pirlindole's central effects via increasing norepinephrine and serotonin enhance auditory processing. The antioxidant activity of pirlindole provide protection to inner ear hair cells from damage caused by reactive oxidative species. Thus, the invention provides a method of preventing or treating hearing loss, an auditory disorder, such as tinnitus, or both in a mammal, such as a human. The method comprises administering a therapeutic amount of a combination of pirlindole and nicergoline, MMDL and/or MDL. to the mammal.

In some embodiments, the method can also include co-administering a therapeutically effective amount of a combination of pirlindole, an NMDA antagonist and one or more of nicergoline, MMDL or MDL. Antagonists of N-methyl-D-aspartate, such as magnesium, riluzole, caroverine and memantine, provide protection against the toxic effects of glutamate, thereby protecting the hair cells of the cochlea of the inner ear from noise-induced damage. Thus, the invention contemplates treatment or prevention of a hearing disorder by administering to a patient an effective amount of a combination of pirlindole, an antagonist of NMDA selected from the group consisting of magnesium, riluzole, caroverine, memantine and combinations thereof, and one or more of nicergoline, MMDL and/or MDL to the patient. In some embodiments, the methods comprise administering to the patient a therapeutic amount of a combination of pirlindole, magnesium and one or more of nicergoline, MMDL and/or MDL. In other particular embodiments, the methods comprise administration of a therapeutic amount of a combination of pirlindole, riluzole and one or more of nicergoline, MMDL and/or MDL. In still further embodiments, the methods comprise administration of a therapeutic amount of a combination of pirlindole, caroverine and one or more of nicergoline, MMDL and/or MDL. In other particular embodiments, the methods comprise administration of a therapeutic amount of a combination of pirlindole, memantine and one or more of nicergoline, MMDL and/or MDL. In other embodiments, the methods comprise administration of a therapeutic amount of a combination of pirlindole, two or more members of the group consisting of magnesium, riluzole, caroverine and memantine, and one or more of nicergoline, MMDL and/or MDL.

8. Nicergoline, MMDL and/or MDL and Milnacipran or Bicifadine

The invention provides methods of preventing or treating a hearing disorder in a mammal, such as a human, using: milnacipran in combination with nicergoline, MMDL and/or MDL; bicifadine in combination with nicergoline, MMDL and/or MDL; or milnacipran and bicifadine in combination with nicergoline, MMDL and/or MDL,. Milnacipran and bicifadine are norepinephrine-serotonin reuptake inhibitors, weak N-methyl-D-aspartate antagonists, which improve auditory processing (NSRI activity) and provide protection to inner ear hair cells (NMDA antagonist activity). Milnacipran, bicifadine or a combination of both will provide both central and peripheral benefits to the treated mammal. Thus, the invention provides a method of preventing or treating hearing loss, an auditory disorder such as tinnitus, or both in a mammal, such as a human, comprising administering a therapeutic amount of a combination of a member of the group consisting of milnacipran, bicifadine or a combination of both to the mammal and one or more of nicergoline, MMDL and/or MDL.

In some embodiments, the method comprises administering to the mammal nicergoline, MMDL and/or MDL and a member of the group consisting of milnacipran, bicifadine and a combination of both in the same dosage form. In particular embodiments, nicergoline, MMDL and/or MDL and a member of the group consisting of milnacipran, bicifadine and a combination of both are mixed together. In other embodiments, nicergoline, MMDL and/or MDL and a member of the group consisting of milnacipran, bicifadine and a combination of both are combined with one or more excipients to form a biphasic or multiphasic dosage form, wherein nicergoline, MMDL and/or MDL and a member of the group consisting of milnacipran, bicifadine and a combination of both occupy separate phases.

In other embodiments, nicergoline, MMDL and/or MDL and a member of the group consisting of milnacipran, bicifadine and a combination of both are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of nicergoline, MMDL and/or MDL and a member of the group consisting of milnacipran, bicifadine and a combination of both are provided in a kit, such as is defined in more detail below. In specific embodiments, the separate dosages are provided in a kit including instructions for the administration of amantadine and nicergoline, MMDL and/or MDL for the prevention or treatment of hearing disorders, especially for the prevention or treatment of hearing loss, tinnitus or both.

9. Indeloxazine and Nicergoline, MMDL and/or MDL

The invention provides methods of preventing or treating a hearing disorder in a mammal, such as a human, using indeloxazine, which is a 5-HT serotonin reuptake inhibitor, a norepinephrine reuptake inhibitor, an acetylcholine releaser, and an antagonist of N-methyl-D-aspartate in combination with one or more of: nicergoline, MMDL and/or MDL. The ability of indeloxazine to increase brain'serotonin, norepinephrine, and acetylcholine levels improves auditory processing, speech recognition and hearing perception. The ability of indeloxazine to block N-methyl-D-aspartate receptors and to act as an antioxidant provides protection to the inner ear cells. Thus, the invention provides a method of preventing or treating hearing loss, an auditory disorder, such as tinnitus, or both in a mammal, such as a human. The method comprises administering a therapeutic amount of indeloxazine to the mammal.

III. Kits

As discussed above, in some embodiments, two or more active pharmaceutical ingredients may be co-administered to a mammal for the prevention or treatment of hearing disorders. In particular, nicergoline, MMDL and/or MDL may be co-administered with or more additional active pharmaceutical ingredients, such as amantadine, milnacipran, bicifadine, pirlindole, amantadine, indeloxazine, antioxidants or spin trapping agents, NMDA antagonists, SSRI/NMDA antagonist compounds, or combinations of at least one SSRI and at least one NMDA antagonist. In such cases, it is advantageous to make the specific drug combination available in the form of a kit.

In some embodiments, the invention provides a kit including nicergoline, MMDL and/or MDL in a dosage form, especially a dosage form for oral administration. Thus, in some embodiments of the invention, the kit includes one or more doses of nicergoline, MMDL and/or MDL in capsules. In other embodiments, however, the doses of nicergoline, MMDL and/or MDL may be present in a variety of dosage forms, such as tablets, caplets, gel caps, powders for suspension, etc.

A kit according to the invention includes at least two dosage forms, one comprising a first active pharmaceutical ingredient and the other comprising at least a second active pharmaceutical ingredient, other than the first active pharmaceutical ingredient. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In some embodiments, the kit according to the invention includes at least two dosage forms, one comprising nicergoline, MMDL and/or MDL and the other comprising at least one active pharmaceutical ingredient other than nicergoline, MMDL and/or MDL. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In particular embodiments, the kit may advantageously be a blister pack. Blister packs are known in the art, and generally include a clear side having compartments (blisters or bubbles), which separately hold the various doses, and a backing, such as a paper, foil, paper-foil or other backing, which is easily removed so that each dose may be separately extracted from the blister pack without disturbing the other doses. In some embodiments, the kit may be a blister pack in which each day's dose of a first active pharmaceutical ingredient and at least a second active pharmaceutical ingredient are segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. Segregation of the two active pharmaceutical ingredients into separate blisters can be advantageous in that it prevents separate dosage forms (e.g. tablet and capsule) from contacting and damaging one another during shipping and handling. Additionally, the separate dosage forms can be accessed and/or labeled for administration to the patient at different times.

In some embodiments, the kit may be a blister pack in which each day's dose of nicergoline, MMDL and/or MDL and at least one other active pharmaceutical ingredient is segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. For example, when nicergoline, MMDL and/or MDL is to be co-administered with riluzole, a specific number of daily doses may be divided into separate removable daily segments, each segment having at least blister containing nicergoline, MMDL and/or MDL from about 0.5 to about 200, especially about 1 to about 100, and most especially about 5 to about 60 mg of nicergoline, or about 20-200%, especially about 50-150% of the equivalent dose of MMDL or MDL (on a molar basis as compared to nicergoline) and at least one other blister containing riluzole (e.g. a 50 mg tablet of riluzole), with perforations separating the segment from its neighbor or neighbors. Segregation of the two active pharmaceutical ingredients into separate blisters can be advantageous in that it prevents separate dosage forms (e.g. tablet and capsule) from contacting and damaging one another during shipping and handling. Additionally, the separate dosage forms can be accessed and/or labeled for administration to the patient at different times. For example, nicergoline, MMDL and/or MDL may cause drowsiness in some patients, and so may be labeled for nighttime administration, whereas other active pharmaceutical ingredients may promote alertness and so may be labeled for daytime administration.

In other embodiments, the kit may be box having separate compartments with separate lids. For example, a kit may comprise a box having seven compartments, each for a separate day of the week, and each compartment marked to indicate which day of the week it corresponds to. In some specific embodiments, each compartment is further subdivided to permit segregation of one active pharmaceutical ingredient from another. As stated above, such segregation is advantageous in that it prevents damage to the dosage forms and permits dosing at different times and labeling to that effect.

It will be understood that kits according to the present invention include those in which the first active pharmaceutical ingredient is selected from the group consisting of nicergoline, MMDL and/or MDL and the second active pharmaceutical ingredient is selected from the group consisting of antioxidants, serotonin reuptake inhibitor—norepinephrine reuptake inhibitor agents, and antagonists of NMDA. Particular kits combine nicergoline, MMDL and/or MDL and with one or members of the group consisting of alaproclate, allopurinol, amantadine, bicifadine, L-camitine, caroverine, S-citalopram, fluoxetine, glutathione, indeloxazine, magnesium, memantine, methionine, milnacipran, nimodipine, pirlindole, riluzole, sertraline, and/or verapamil.

### EXAMPLES

The invention will now be further illustrated by means of the following, non-limiting examples.

### Example 1

### In Vivo Efficacy of Nicergoline in the Treatment of Hearing Loss

In order to demonstrate the efficacy of nicergoline in the treatment and prevention of drug- and noise-induced hearing loss, nicergoline is evaluated in animal models of drug- and noise-induced hearing loss, as described below.

Experimental Animals

Male CBA mice, at an initial age of 4 weeks, are purchased from Harlan Sprague-Dawley Co. (Indianapolis, Ind.). The animals are given free access to water and a regular mouse diet (Purina, St. Louis, Mo.), and are allowed 1 week to acclimate before treatment.

Drug-Induced Hearing Loss

The aminoglycoside antibiotic kanamycin (700 mg of kanamycin base/kg of body weight twice daily) is tested alone and in combination with various concentrations of nicergoline. The study to evaluate auditory effects is comprised of one group serving as the vehicle control group, one group receiving kanamycin injections only, three groups receiving kanamycin plus nicergoline at 3, 10, or 30 mg/kg twice daily, and three groups receiving nicergoline only at 3, 10, or 30 mg/kg twice daily. In a separate study, levels of kanamycin in serum are determined for animals receiving kanamycin alone (700 mg of kanamycin base/kg twice daily) or kanamycin plus nicergoline (10 or 30 mg/kg twice daily). Kanamycin and nicergoline are each dissolved in suitable vehicle, and injections are given separately but simultaneously twice daily for 15 days. Control mice receive an equivalent volume of vehicle.

Noise-Induced Hearing Loss

Mice are exposed to a 4kHz octave band of noise at 110 dB SPL for 4 h and receive treatment with vehicle or nicergoline (3, 10, or 30 mg/kg) twice daily beginning 3 days prior and continuing until 1 day following noise exposure.

Evaluation of Auditory Function

Auditory thresholds are measured by evoked auditory brainstem responses (ABR). Thresholds are taken for each animal at the beginning of the study for all animals. For animals treated with kanamycin, ABR measurements are also assessed 2 weeks after the start of drug treatment, and then at 3 weeks and 5 weeks. For animals exposed to noise, ABR measurements are determined prior to and 10 days after noise exposure. The mice are anesthetized with an intramuscular injection of 100 mg of ketamine and 5 mg of xylazine/kg and kept warm with a heating pad. The positive needle electrode is sub-dermally inserted at the vertex, the midline of the scalp between the external auditory canals. The negative electrode is placed below the pinna of the left ear, and the ground electrode is inserted contralaterally. Tone bursts of 12 and 24 kHz (10 ms duration, 1 ms rise and fall time) are generated using a SigGen software package (Tucker-Davis Technologies, Gainesville, Fla.) and are delivered to the left external auditory meatus in a closed acoustic system through an ear bar connected to a DT-48 transducer (Beyer Dynamic, Farmingdale, N.Y.). Responses from 1020 stimuli are averaged for each frequency, fed to an amplifier, viewed with an oscilloscope, and recorded. Threshold is determined by reducing the sound intensity in 5 dB steps until threshold. Thresholds are defined as the lowest intensity that yields a reproducible deflection in the evoked response trace and are verified at least twice. Threshold shifts are calculated for individual animals by comparison to their pre-study thresholds. The ABR of each animal is interpreted by an observer without knowledge of the treatment.

Hair Cell Counts

After the last ABR measurement, animals are sacrificed, and the temporal bones are removed. The round and oval windows and the apex of the cochlea are opened, perfused with 4% paraformaldehyde in 10 mM phosphate-buffered saline (pH 7.4), and fixed overnight at 4° C. The fixed cochleae are decalcified with 4% EDTA in 10 mM phosphate-buffered saline for 2 to 3 days. After removal of the bony capsule, lateral wall, and tectorial membrane, cochleae are stained with rhodamine phalloidin for 50 min to outline the hair cells. Thereafter, the organ of Corti is separated from the modiolus, microdissected into individual turns, mounted on glass slides in antifade fluorescent mounting media, and coverslipped.

Hair cells in the organ of Corti are counted on a Leitz Orthoplan upright fluorescence microscope, using a x50 oil immersion objective lens. Evaluation begins at the apex and moves down the cochlear spiral to the base, assessing successive 0.19-mm fields. For each field, the area of observation is oriented to include the row of inner hair cells and all three rows of outer hair cells longitudinally. After counting of the entire cochlea, each row is evaluated for the presence or absence of hair cells. Manual count data are entered into a computer program. The percentage of missing hair cells for each row is calculated, utilizing normative counts from control animals as 100%, and cytocochleograms are plotted for the percentage of cell loss.

### Example 2

### In Vivo Efficacy of MMDL and MDL in the Treatment of Hearing Loss

In order to demonstrate the efficacy of MMDL and MDL in the treatment and prevention of drug- and noise-induced hearing loss, MMDL and MDL are evaluated in animal models of drug- and noise-induced hearing loss, as described in Example 1, above.

Preparation of MMDL and MDL

MMDL ((8β)-10-methoxy-1,6-dimethylergoline-8-methanol) can be prepared from nicergoline by hydrolysis of the 5-bromo-3-pyridine carboxylate group from the 8-methanol moiety by a method known in the art. In general, hydrolysis may be accomplished by treatment with acid or base. Heat may be applied to increase the rate of the reaction. Workup may be accomplished by a suitable separation method, such as preparative scale high performance liquid chromatography, thin layer chromatography, separation in a separatory funnel, or other art-recognized method.

MDL ((8β-10-methoxy-6-methylergoline-8-methanol) may be prepared form nicergoline by removing the N1-methyl group from MMDL by an art-recognized method. In particular, MMDL has been shown to be converted into MDL by CYP2D6 (dextromethorphan 0-demethylase). See Bottiger et al., “Involvement of CYP2D6 but not CYP2C19 in nicergoline metabolism in humans,” Br. J. Clin. Pharmacol., 42, 707-711 (1996). Thus, MDL may be prepared in vitro from MMDL starting material by contacting a solution containing MMDL with isolated CYP2D6, human liver microsomal preparations expressing CYP2D6 or transformed cells expressing CYP2D6. Suitable human liver microsomes are described in the literature. See, Lillibridge et al., “Characterization of the Selectivity and Mechanism of Human Cytochrome P450 Inhibition by the Human Immunodeficiency Virus-Protease Inhibitor Nelfinavir Mesylate,” Drug Metabolism and Disposition, 26(7), 609-616 (1998). Isolation of MDL may be effected by analytical or preparative scale high performance chromatography or other suitable separation method.

### Example 3

### Cochlear Cultures

Explants of sensory epithelium from the basal turn of postnatal day 5 (p5) Sprague-Dawley rats were isolated using the methods of Van De Water and Sobkowicz (Van d Water T., Ruben R. J. (1974) “Growth of the inner ear in organ culture,” Ann. Otol. Rhinol. Laryngol., 83:1-16; Sobkowicz H. M., Loftus J. M., Slapnick S. M. (1993) “Tissue culture of the organ of Corti.,” Acta. Suppl. 502:3-36). Explants were maintained in Dulbecco's Modified Eagle's medium with 10% fetal bovine serum and 30 U/ml penicillin. HEPES buffer was added to a concentration of 25 mM and the glucose concentration increased to 6 mg/ml to enhance neuronal survival. Each 15 mm dish containing 250 μl of medium was maintained in an incubator at 37° C. with 5% CO₂ and 95% humidity.

Toxicity-inducers (gentamicin or cisplatin, 1-100 μM) were exposed to the medium 12-24 h following plating for 48 h. Concentrations of gentamicin or cisplatin were employed that produced approximately 50% loss of outer hair cells, which permitted for assessments of either increases or decreases in outer hair cell damage.

Test compounds were applied to the cultures for 12 h, and then challenged with medium containing gentamicin or cisplatin plus the test compound for an additional 48 h, followed by fixation and evaluation.

At the end of culture, free floating explants were fixed with 4% paraformaldehyde and 1% glutaraldehyde in phosphate-buffered saline. The explants were then permeabilized with 1 % Tween-20 in phosphate-buffered saline. The explants were then stained with conjugated phalloidin-rhodamine probe in phosphate-buffered saline, washed twice, and mounted between two cover slips in a 1:3 mixture of phosphate-buffered saline/glycerol. Explants were imaged by light and fluorescence microscopy and by laser confocal microscopy to assess the degree of damage to the outer hair cells.

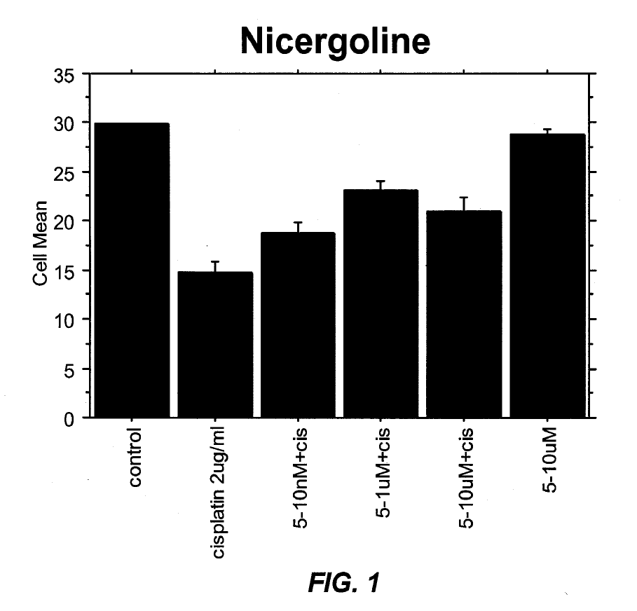

The results of one such test, employing cisplatin as an ototoxic agent and nicergoline as a test compound, are shown in FIG. 1. The control cultures contained no cisplatin and no nicergoline. Cultures containing 2 μg/ml cisplatin and 0 nM, 10 nM, 1 μM or 10 μM nicergoline (5 cultures each) were also tested, as were 5 cultures containing 0 μg/ml cisplatin and 10 μM nicergoline. As can be seen from FIG. 1, nicergoline itself is non-toxic to cochlear cultures at concentrations up to 10 μM; and nicergoline provides maximum protection from 2 μM cisplatin-induced cell death at a concentration of about 1 μM. Thus, nicergoline is non-toxic to cochlear cells, while exerting protection against the toxic effects of cisplatin at all tested concentrations.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A method of preventing or treating a hearing disorder in a mammal, comprising administering to the mammal a therapeutic amount of a therapeutic composition comprising nicergoline, MMDL or MDL, or a pharmaceutically acceptable salt or polymorph thereof.

2. The method of claim 1, wherein the therapeutic composition comprises nicergoline or a pharmaceutically acceptable salt or polymorph thereof.

3. The method of claim 1, wherein the therapeutic composition comprises MMDL or a pharmaceutically acceptable salt or polymorph thereof.

4. The method of claim 1, wherein the therapeutic composition comprises MDL or a pharmaceutically acceptable salt or polymorph thereof.

5. The method of claim 1, wherein the mammal is a human.

6. The method of claim 1, wherein the therapeutic composition comprises a detectable amount of only one of nicergoline, MMDL or MDL or a pharmaceutically acceptable salt or polymorph thereof.

7. The method of claim 1, wherein the therapeutic composition consists essentially of nicergoline, MMDL or MDL, or a combination thereof, in admixture with one or more physiologically acceptable excipients.

8. The method of claim 1, wherein the therapeutic composition further comprises one or more antioxidants, spin-trapping agents, or both.

9. The method of claim 1, wherein the therapeutic composition further comprises one or more antioxidants.

10. The method of claim 1, wherein the therapeutic composition further comprises one or more compound having norepinephrine reuptake inhibiting and serotonin reuptake inhibiting activity.

11. The method of claim 10, wherein at least one compound having norepinephrine reuptake inhibiting and serotonin reuptake inhibiting activity is selected from the group consisting of milnacipran and bicifadine.

12. The method of claim 1, wherein the therapeutic composition further comprises one or more antioxidants selected from allopurinol, glutathione, methionine, L-camitine, ebselen, AEOL-10150 and mixtures of two or more thereof.

13. The method of claim 12, wherein the antioxidant is a mixture of two or more of allopurinol, glutathione, methionine and L-camitine.

14. The method of claim 1 wherein the hearing disorder is selected from the group consisting of noise-induced hearing loss, drug-induced hearing loss, presbyacusis, central auditory processing disorder and tinnitus.

15. The method of claim 14, wherein the hearing disorder is drug-induced hearing loss.

16. The method of claim 15, wherein the therapeutic composition is administered prior to administration of an ototoxic drug, whereby drug-induced hearing loss is at least partially prevented.

17. The method of claim 15, wherein the therapeutic composition is administered at substantially the same time as or after administration of an ototoxic drug, whereby drug-induced hearing loss is prevented, reduced, ameliorated or reversed.

18. The method of claim 1, wherein the therapeutic composition further comprises one or more agents that bind to or metabolize reactive oxygen species and provide protection against the damage induced by toxic oxygen mediator species.

19. The method of claim 1, wherein the therapeutic composition further comprises at least one N-methyl-D-aspartate antagonist.

20. The method of claim 19, wherein the therapeutic composition further comprises at least one N-methyl-D-aspartate antagonist selected from the group consisting of magnesium, riluzole, caroverine, memantine and mixtures thereof.

21. The method of claim 1, wherein the therapeutic composition further comprises an agent that blocks the excitotoxic actions of glutamate within the inner ear, whereby the noise-induced damage mediating effects of glutamate are blocked, and protection of the hair cells within the cochlea of the inner ear is effected.

22. The method of claim 1, wherein the therapeutic composition further comprises a compound having selective serotonin reuptake inhibitor activity and N-methyl-D-aspartate antagonist activity.

23. The method of claim 1, wherein the therapeutic composition further comprises a compound having selective serotonin reuptake inhibitor activity and a compound having N-methyl-D-aspartate antagonist activity.

24. The method of claim 1, wherein the therapeutic composition further comprises a selective serotonin reuptake inhibitor selected from fluoxetine, sertraline, S-citalopram and mixtures thereof and a N-methyl-D-aspartate antagonist selected from magnesium, riluzole, caroverine, memantine and mixtures thereof.

25. The method of claim 1, wherein the therapeutic composition further comprises a first compound that enhances the synaptic levels of serotonin in the brain and enhances hearing, thereby improving auditory processing, increasing the signal: noise ratio of environmental sounds or heightening attention; and a second compound that blocks the excitotoxic actions of glutamate in the inner ear, thereby blocking the glutamate-mediated noise-induced damage to the hair cells of the inner ear.

26. The method of claim 1, further comprising administering to the mammal one other active compound.

27. The method of claim 1, further comprising administering to the mammal an antioxidant.

28. The method of claim 1, further comprising administering to the mammal an antioxidant selected from allopurinol, glutathione, methionine, L-camitine and mixtures thereof.

29. The method of claim 1, further comprising administering to the mammal at least one compound having norepinephrine reuptake inhibiting and serotonin reuptake inhibiting activity.

30. The method of claim 29, wherein a compound having norepinephrine reuptake inhibiting and serotonin reuptake inhibiting activity is selected from the group consisting of milnacipran and bicifadine.

31. The method of claim 1, further comprising administering to the mammal at least one other active compound that binds to or metabolizes reactive oxygen species and provides protection against oxygen species-induced damage to the hair cells of the cochlea of the inner ear.

32. The method of claim 1, further comprising administering to the mammal at least one N-methyl-D-aspartate antagonist.

33. The method of claim 1, further comprising administering to the mammal at least one N-methyl-D-aspartate antagonist selected from the group consisting of magnesium, riluzole, caroverine, memantine and mixtures thereof.

34. The method of claim 1, further comprising administering to the mammal at least one additional active compound, wherein the additional active compound blocks the excitotoxic actions of glutamate within the inner ear, thereby blocking the glutamate-mediated noise-induced damage to the hair cells of the cochlea of the inner ear.

35. The method of claim 1, further comprising administering to the mammal at least one additional active compound, wherein at least one additional active compound has selective serotonin reuptake inhibitory and N-methyl-D-aspartate agonist activity.

36. The method of claim 1, further comprising administering to the mammal at least one selective serotonin reuptake inhibitor and at least one N-methyl-D-aspartate antagonist.

37. The method of claim 36, wherein the selective serotonin reuptake inhibitor is selected from the group consisting of fluoxetine, sertraline, S-citalopram and mixtures of two or more thereof.

38. The method of claim 36, wherein the N-methyl-D-aspartate antagonist is selected from the group consisting of magnesium, riluzole, caroverine, memantine and mixtures thereof.

39. The method of claim 36, wherein the selective serotonin reuptake inhibitor is selected from the group consisting of fluoxetine, sertraline, S-citalopram and mixtures of two or more thereof

40. The method of claim 1, further comprising administering to the mammal a compound that enhances the synaptic levels of serotonin in the brain and enhancing hearing, thereby improving auditory processing, increasing the signal: noise ratio of environmental sounds or heightening attention and a compound that blocks the excitotoxic actions of glutamate in the inner ear, thereby blocking the glutamate-mediated noise-induced damage to the hair cells of the inner ear.

41. A method of claim 1, wherein the hearing disorder is a central auditory processing disorder.

42. A method of claim 41, wherein the therapeutic composition further comprises one or more additional active ingredients.

43. The method of claim 42, wherein the therapeutic composition further comprises one or more additional active ingredients selected from the group consisting of antioxidants, norepinephrine reuptake inhibitors, serotonin reuptake inhibitors, norepinephrine-epinephrine reuptake inhibitors, and combinations thereof.

44. The method of claim 42, further comprising administering to the patient a separate dosage form containing one or more members of the group consisting of active ingredients selected from the group consisting of antioxidants, norepinephrine reuptake inhibitors, serotonin reuptake inhibitors, norepinephrine-epinephrine reuptake inhibitors, and combinations thereof.

45. A therapeutic composition for treating or preventing hearing loss in a mammal comprising a therapeutic amount of nicergoline, MMDL or MDL.

46. The therapeutic composition of claim 45, wherein the therapeutic composition comprises nicergoline.

47. The therapeutic composition of claim 45, wherein the therapeutic composition comprises MMDL.

48. The therapeutic composition of claim 45, wherein the therapeutic composition comprises MDL.

49. The therapeutic composition of claim 45, wherein the mammal is a human.

50. The therapeutic composition of claim 45, wherein the therapeutic composition consists essentially of nicergoline, MMDL or MDL in admixture with one or more physiologically acceptable excipients.

51. The therapeutic composition of claim 45, wherein the therapeutic composition further comprises one or more antioxidants, spin-trapping agents, or both.

52. The therapeutic composition of claim 51, wherein the therapeutic composition comprises one or more antioxidants.

53. The therapeutic composition of claim 5 1, wherein the therapeutic composition comprises one or more antioxidants selected from allopurinol, glutathione, methionine, L-camitine and mixtures of two or more thereof.

54. The therapeutic composition of claim 53, wherein the antioxidant is a mixture of two or more of allopurinol, glutathione, methionine and L-camitine.

55. The therapeutic composition of claim 45, wherein the therapeutic composition further comprises one or more agents that bind to or metabolize reactive oxygen species and provide protection against the damage induced by toxic oxygen mediator species.

56. The therapeutic composition of claim 45, wherein the therapeutic composition further comprises at least one NMDA antagonist.

57. The therapeutic composition of claim 56, wherein the therapeutic composition comprises at least one NMDA antagonist selected from the group consisting of magnesium, riluzole, caroverine, memantine and mixtures thereof.

58. The therapeutic composition of claim 45, wherein the therapeutic composition further comprises an agent that blocks the excitotoxic actions of glutamate within the inner ear, whereby the noise-induced damage mediating effects of glutamate are blocked, and protection of the hair cells within the cochlea of the inner ear is effected.

59. The therapeutic composition of claim 45, wherein the therapeutic composition further comprises a compound having selective serotonin reuptake inhibitor activity and N-methyl-D-aspartate antagonist activity.

60. The therapeutic composition of claim 45, wherein the therapeutic composition further comprises a compound having selective serotonin reuptake inhibitor activity and a compound having N-methyl-D-aspartate antagonist activity.

61. The therapeutic composition of claim 45, wherein the therapeutic composition further comprises a selective serotonin reuptake inhibitor selected from fluoxetine, sertraline, S-citalopram and mixtures thereof and a N-methyl-D-aspartate antagonist selected from magnesium, riluzole, caroverine, memantine and mixtures thereof.

62. The therapeutic composition of claim 45, wherein the therapeutic composition further comprises one or more compounds having norepinephrine reuptake inhibiting and serotonin reuptake inhibiting activity.

63. The therapeutic composition of claim 62, wherein at least one compound having norepinephrine reuptake inhibiting and serotonin reuptake inhibiting activity is selected from the group consisting of milnacipran and bicifadine.

64. A kit for treating or preventing hearing loss in a mammal, comprising nicergoline, MMDL or MDL and at least one other active pharmaceutical ingredient.

65. The kit of claim 64, wherein nicergoline, MMDL or MDL and the other active compound are compounded in the same dose.

66. The kit of claim 64, wherein nicergoline, MMDL or MDL and the other active compound are compound in separate doses.

67. Kit of claim 64, comprising nicergoline, MMDL or MDL and an antioxidant.

68. The kit of claim 64, comprising nicergoline, MMDL or MDL and an antioxidant selected from allopurinol, glutathione, methionine, L-camitine and mixtures thereof.

69. The kit of claim 64, wherein the nicergoline, MMDL or MDL and the antioxidant are compounded in the same dose.

70. The kit of claim 64, wherein the nicergoline, MMDL or MDL and the antioxidant are compounded in separate doses.

71. The kit of claim 64, comprising nicergoline, MMDL or MDL and at least one other active compound, wherein at least one other active compound binds to or metabolizes reactive oxygen species and provides protection against oxygen species-induced damage to the hair cells of the cochlea of the inner ear.

72. The kit of claim 64, comprising nicergoline, MMDL or MDL and at least one N-methyl-D-aspartate antagonist.

73. The kit of claim 64, comprising nicergoline, MMDL or MDL and at least one N-methyl-D-aspartate antagonist selected from the group consisting of magnesium, riluzole, caroverine, memantine and mixtures thereof.

74. The kit of claim 64, comprising nicergoline, MMDL or MDL and at least one compound having selective serotonin reuptake inhibitory and N-methyl-D-aspartate agonist activity.

75. The kit of claim 64, comprising nicergoline, MMDL or MDL and at least one selective serotonin reuptake inhibitor and at least one N-methyl-D-aspartate antagonist.

76. The kit of claim 75, wherein the selective serotonin reuptake inhibitor is selected from the group consisting of fluoxetine, sertraline, S-citalopram and mixtures of two or more thereof.

77. The kit of claim 75, wherein the N-methyl-D-aspartate antagonist is selected from the group consisting of magnesium, riluzole, caroverine, memantine and mixtures thereof.

78. The kit of claim 64, comprising a first compound that enhances the synaptic levels of serotonin in the brain and enhances hearing, thereby improving auditory processing, increasing the signal: noise ratio of environmental sounds or heightening attention; and a second compound that blocks the excitotoxic actions of glutamate in the inner ear, thereby blocking the glutamate-mediated noise-induced damage to the hair cells of the inner ear.

79. The kit of claim 64, comprising a nicergoline, MMDL or MDL and having norepinephrine reuptake inhibiting and serotonin reuptake inhibiting activity.

80. The kit of claim 64, wherein at least one compound having norepinephrine reuptake inhibiting and serotonin reuptake inhibiting activity is selected from the group consisting of milnacipran and bicifadine.

81. The kit of claim 64, wherein the nicergoline, MMDL or MDL and at least one compound having norepinephrine reuptake inhibiting and serotonin reuptake inhibiting activity are in separate doses.

82. The kit of claim 64, wherein the nicergoline, MMDL or MDL and at least one compound having norepinephrine reuptake inhibiting and serotonin reuptake inhibiting activity are in the same dose.

83. A method of treating or preventing a hearing disorder in a human patient, comprising administering to the human patient a therapeutically effective amount of a therapeutic composition comprising a nicergoline metabolite MDL, wherein the therapeutically effective amount of the therapeutic composition is effective to treat or prevent at least one hearing disorder.

84. The method of claim 83, wherein the therapeutic composition consists essentially of MDL and one or more diluents or excipients.

85. The method of claim 83, wherein the therapeutic composition further comprises one or more antioxidants, spin-trapping agents, or both.

86. The method of claim 83, wherein the therapeutic composition further comprises one or more antioxidants.

87. The method of claim 83, wherein the therapeutic composition further comprises one or more compound having norepinephrine reuptake inhibiting and serotonin reuptake inhibiting activity.

88. The method of claim 88, wherein at least one compound having norepinephrine reuptake inhibiting and serotonin reuptake inhibiting activity is selected from the group consisting of milnacipran and bicifadine.

89. The method of claim 83, wherein the therapeutic composition further comprises one or more antioxidants selected from allopurinol, glutathione, methionine, L-camitine, ebselen, AEOL-10150 and mixtures of two or more thereof.

90. The method of claim 89, wherein the antioxidant is a mixture of two or more of allopurinol, glutathione, methionine and L-camitine.

91. The method of claim 83, wherein the hearing disorder is selected from the group consisting of noise-induced hearing loss, drug-induced hearing loss, presbyacusis, central auditory processing disorder and tinnitus.

92. The method of claim 91, wherein the hearing disorder is drug-induced hearing loss.

93. The method of claim 92, wherein the therapeutic composition is administered prior to administration of an ototoxic drug, whereby drug-induced hearing loss is at least partially prevented.

94. The method of claim 93, wherein the therapeutic composition is administered at substantially the same time as or after administration of an ototoxic drug, whereby drug-induced hearing loss is reduced, ameliorated or reversed.

95. The method of claim 83, wherein the therapeutic composition further comprises one or more agents that bind to or metabolize reactive oxygen species and provide protection against the damage induced by toxic oxygen mediator species.

96. The method of claim 83, wherein the therapeutic composition further comprises at least one N-methyl-D-aspartate antagonist.

97. The method of claim 96, wherein the therapeutic composition further comprises at least one N-methyl-D-aspartate antagonist selected from the group consisting of magnesium, riluzole, caroverine, memantine and mixtures thereof.

98. The method of claim 83, wherein the therapeutic composition further comprises an agent that blocks the excitotoxic actions of glutamate within the inner ear, whereby the noise-induced damage mediating effects of glutamate are blocked, and protection of the hair cells within the cochlea of the inner ear is effected.

99. The method of claim 83, wherein the therapeutic composition further comprises a compound having selective serotonin reuptake inhibitor activity and a compound having N-methyl-D-aspartate antagonist activity.

100. The method of claim 83, wherein the therapeutic composition further comprises a compound having selective serotonin reuptake inhibitor activity and N-methyl-D-aspartate antagonist activity.

101. The method of claim 83, wherein the therapeutic composition further comprises a selective serotonin reuptake inhibitor selected from fluoxetine, sertraline, S-citalopram and mixtures thereof and a N-methyl-D-aspartate antagonist selected from magnesium, riluzole, caroverine, memantine and mixtures thereof.

102. The method of claim 83, wherein the therapeutic composition further comprises a first compound that enhances the synaptic levels of serotonin in the brain and enhances hearing, thereby improving auditory processing, increasing the signal: noise ratio of environmental sounds or heightening attention; and a second compound that blocks the excitotoxic actions of glutamate in the inner ear, thereby blocking the glutamate-mediated noise-induced damage to the hair cells of the inner ear.

103. The method of claim 83, further comprising administering to the mammal at least one active compound other than a member of the group consisting of nicergoline, MMDL and MDL.

104. The method of claim 103, further comprising administering to the mammal an antioxidant.

105. The method of claim 104, further comprising administering to the mammal an antioxidant selected from allopurinol, glutathione, methionine, L-carnitine and mixtures thereof.

106. The method of claim 83, further comprising administering to the mammal at least one compound having norepinephrine reuptake inhibiting and serotonin reuptake inhibiting activity.

107. The method of claim 106, wherein a compound having norepinephrine reuptake inhibiting and serotonin reuptake inhibiting activity is selected from the group consisting of milnacipran and bicifadine.

108. The method of claim 83, further comprising administering to the mammal at least one other active compound that binds to or metabolizes reactive oxygen species and provides protection against oxygen species-induced damage to the hair cells of the cochlea of the inner ear.

109. The method of claim 83, further comprising administering to the mammal at least one N-methyl-D-aspartate antagonist.

110. The method of claim 83, further comprising administering to the mammal at least one N-methyl-D-aspartate antagonist selected from the group consisting of magnesium, riluzole, caroverine, memantine and mixtures thereof.

111. The method of claim 83, further comprising administering to the mammal at least one additional active compound, wherein the additional active compound blocks the excitotoxic actions of glutamate within the inner ear, thereby blocking the glutamate-mediated noise-induced damage to the hair cells of the cochlea of the inner ear.

112. The method of claim 83, further comprising administering to the mammal at least one additional active compound, wherein at least one additional active compound has selective serotonin reuptake inhibitory and N-methyl-D-aspartate agonist activity.

113. The method of claim 83, further comprising administering to the mammal at least one selective serotonin reuptake inhibitor and at least one N-methyl-D-aspartate antagonist.

114. The method of claim 1 13, wherein the selective serotonin reuptake inhibitor is selected from the group consisting of fluoxetine, sertraline, S-citalopram and mixtures of two or more thereof.

115. The method of claim 113, wherein the N-methyl-D-aspartate antagonist is selected from the group consisting of magnesium, riluzole, caroverine, memantine and mixtures thereof.

116. The method of claim 113, wherein the selective serotonin reuptake inhibitor is selected from the group consisting of fluoxetine, sertraline, S-citalopram and mixtures of two or more thereof

117. The method of claim 83, further comprising administering to the mammal a compound that enhances the synaptic levels of serotonin in the brain and enhancing hearing, thereby improving auditory processing, increasing the signal: noise ratio of environmental sounds or heightening attention and a compound that blocks the excitotoxic actions of glutamate in the inner ear, thereby blocking the glutamate-mediated noise-induced damage to the hair cells of the inner ear.

118. A method of claim 83, wherein the hearing disorder is a central auditory processing disorder.

119. A method of claim 118, wherein the therapeutic composition further comprises one or more additional active ingredients.

120. The method of claim 119, wherein the therapeutic composition further comprises one or more additional active ingredients selected from the group consisting of antioxidants, norepinephrine reuptake inhibitors, serotonin reuptake inhibitors, norepinephrine-epinephrine reuptake inhibitors, and combinations thereof.

121. The method of claim 120, further comprising administering to the patient a separate dosage form containing one or more members of the group consisting of active ingredients selected from the group consisting of antioxidants, norepinephrine reuptake inhibitors, serotonin reuptake inhibitors, norepinephrine-epinephrine reuptake inhibitors, and combinations thereof.

122. The method of claim 83, wherein the human patient is substantially deficient in expression of the CYP2D6 demethylase.

123. The method of claim 83, wherein the method decreases the variability associated with CYP2D6 polymorphism and thereby increases the efficacy of nicergoline therapy in humans.
